SEQUENCE LISTING (108 sequences)

SEQ ID NO: 1 - DNA, 22 bp, Artificial sequence
  Synthetic
tgaccacttctgtgctggatgg

SEQ ID NO: 2 - DNA, 22 bp, Artificial sequence
  Synthetic
agcaagctctccaccatagcca

SEQ ID NO: 3 - DNA, 22 bp, Artificial sequence
  Synthetic
atcctggaggaccagaactgca

SEQ ID NO: 4 - DNA, 22 bp, Artificial sequence
  Synthetic
ggcgaggctcttagatttctcc

SEQ ID NO: 5 - DNA, 22 bp, Artificial sequence
  Synthetic
gaggtgcttcagaatgaggacg

SEQ ID NO: 6 - DNA, 22 bp, Artificial sequence
  Synthetic
gcatcagtgacacctggcaact

SEQ ID NO: 7 - DNA, 22 bp, Artificial sequence
  Synthetic
cggtgaatctgggcttatggga

SEQ ID NO: 8 - DNA, 23 bp, Artificial sequence
  Synthetic
ggaggttatatccttaccgtacg

SEQ ID NO: 9 - DNA, 22 bp, Artificial sequence
  Synthetic
tctgtcccaacgtgcccatcat

SEQ ID NO: 10 - DNA, 22 bp, Artificial sequence
  Synthetic
ctgccttcttcaggtttcaccg

SEQ ID NO: 11 - DNA, 23 bp, Artificial sequence
  Synthetic
gaaacagtgttccatgctagacg

SEQ ID NO: 12 - DNA, 23 bp, Artificial sequence
  Synthetic
gccgcttatttgattcctgctcc

SEQ ID NO: 13 - DNA, 22 bp, Artificial sequence
  Synthetic
gtgaaggctgtgtctgagactc

SEQ ID NO: 14 - DNA, 23 bp, Artificial sequence
  Synthetic
ggaagtggttcaatcacagaccg

SEQ ID NO: 15 - DNA, 22 bp, Artificial sequence
  Synthetic
cgactccaacacagtgaagcag

SEQ ID NO: 16 - DNA, 22 bp, Artificial sequence
  Synthetic
tcactactgcgggtgcttctgt

SEQ ID NO: 17 - DNA, 22 bp, Artificial sequence
  Synthetic
ctacgagtggatggtcaagagc

SEQ ID NO: 18 - DNA, 23 bp, Artificial sequence
  Synthetic
ccagttccttcattctgcacacg

SEQ ID NO: 19 - DNA, 23 bp, Artificial sequence
  Synthetic
gcttgctggtttgtcatcactcc

SEQ ID NO: 20 - DNA, 22 bp, Artificial sequence
  Synthetic
tacaccagccaagagcaactcc

SEQ ID NO: 21 - DNA, 23 bp, Artificial sequence
  Synthetic
cactaccaacctcatggaagagg

SEQ ID NO: 22 - DNA, 22 bp, Artificial sequence
  Synthetic
tccaactcctgcctctgcttct

SEQ ID NO: 23 - DNA, 22 bp, Artificial sequence
  Synthetic
cactgtcacctggtcgctgaat

SEQ ID NO: 24 - DNA, 22 bp, Artificial sequence
  Synthetic
gcgctgttcttggctacacact

SEQ ID NO: 25 - DNA, 23 bp, Artificial sequence
  Synthetic
ggacaccattgagaagctgaagg

SEQ ID NO: 26 - DNA, 22 bp, Artificial sequence
  Synthetic
gtcttgagtcctcgctgtgtga

SEQ ID NO: 27 - DNA, 22 bp, Artificial sequence
  Synthetic
cttcagcaaggaccagttccca

SEQ ID NO: 28 - DNA, 22 bp, Artificial sequence
  Synthetic
ggcggtcataatcttcctgtcc

SEQ ID NO: 29 - DNA, 22 bp, Artificial sequence
  Synthetic
cacgcctaactgacaagcacca

SEQ ID NO: 30 - DNA, 23 bp, Artificial sequence
  Synthetic
caggtcaacatctagcatggaac

SEQ ID NO: 31 - DNA, 22 bp, Artificial sequence
  Synthetic
attgctccacgcccagaacaca

SEQ ID NO: 32 - DNA, 22 bp, Artificial sequence
  Synthetic
aagcatctggcggagtggtgtt

SEQ ID NO: 33 - DNA, 23 bp, Artificial sequence
  Synthetic
gctgtagtgacagaggaagatga

SEQ ID NO: 34 - DNA, 22 bp, Artificial sequence
  Synthetic
tcaccaactggagcaggaatgg

SEQ ID NO: 35 - DNA, 22 bp, Artificial sequence
  Synthetic
ctacgagtggatggtgaagagc

SEQ ID NO: 36 - DNA, 23 bp, Artificial sequence
  Synthetic
ccagttccttcattctgcactcg

SEQ ID NO: 37 - DNA, 15 bp, Artificial sequence
  Synthetic
ccaccacgtccagct

SEQ ID NO: 38 - DNA, 25 bp, Artificial sequence
  Synthetic
ccctgtttctggaaaaaataaaaat

SEQ ID NO: 39 - DNA, 25 bp, Artificial sequence
  Synthetic
atttttattttttccagaaacaggg

SEQ ID NO: 40 - DNA, 24 bp, Artificial sequence
  Synthetic
ggaagagcaaatttatagaatgca

SEQ ID NO: 41 - DNA, 24 bp, Artificial sequence
  Synthetic
agcattctataaatttgctcttcc

SEQ ID NO: 42 - DNA, 20 bp, Artificial sequence
  Synthetic
aagaccatcctgggtaacat

SEQ ID NO: 43 - DNA, 20 bp, Artificial sequence
  Synthetic
atgttacccaggatggtctt

SEQ ID NO: 44 - DNA, 25 bp, Artificial sequence
  Synthetic
gttctttgcaacattgtttatagtc

SEQ ID NO: 45 - DNA, 25 bp, Artificial sequence
  Synthetic
gactataaacaatgttgcaaagaac

SEQ ID NO: 46 - DNA, 19 bp, Artificial sequence
  Synthetic
caggagatcgagaccatcc

SEQ ID NO: 47 - DNA, 19 bp, Artificial sequence
  Synthetic
ggatggtctcgatctcctg

SEQ ID NO: 48 - DNA, 23 bp, Artificial sequence
  Synthetic
aaaaagccataagaaatgcaaac

SEQ ID NO: 49 - DNA, 23 bp, Artificial sequence
  Synthetic
gtttgcatttcttatggcttttt

SEQ ID NO: 50 - DNA, 18 bp, Artificial sequence
  Synthetic
gctgaggcaggagaatcg

SEQ ID NO: 51 - DNA, 18 bp, Artificial sequence
  Synthetic
cgattctcctgcctcagc

SEQ ID NO: 52 - DNA, 24 bp, Artificial sequence
  Synthetic
aactctgtgacaaattactatcag

SEQ ID NO: 53 - DNA, 24 bp, Artificial sequence
  Synthetic
ctgatagtaatttgtcacagagtt

SEQ ID NO: 54 - DNA, 21 bp, Artificial sequence
  Synthetic
acttgaagctgtttctgactt

SEQ ID NO: 55 - DNA, 21 bp, Artificial sequence
  Synthetic
aagtcagaaacagcttcaagt

SEQ ID NO: 56 - DNA, 22 bp, Artificial sequence
  Synthetic
catgtcattaactcgcatacat

SEQ ID NO: 57 - DNA, 22 bp, Artificial sequence
  Synthetic
atgtatgcgagttaatgacatg

SEQ ID NO: 58 - DNA, 18 bp, Artificial sequence
  Synthetic
catcccaccacaaggaca

SEQ ID NO: 59 - DNA, 18 bp, Artificial sequence
  Synthetic
tgtccttgtggtgggatg

SEQ ID NO: 60 - DNA, 16 bp, Artificial sequence
  Synthetic
tctagggcgggtccaa

SEQ ID NO: 61 - DNA, 16 bp, Artificial sequence
  Synthetic
ttggacccgccctaga

SEQ ID NO: 62 - DNA, 15 bp, Artificial sequence
  Synthetic
ggggcacaatcccgc

SEQ ID NO: 63 - DNA, 22 bp, Artificial sequence
  Synthetic
tctagtagaggacaagctttca

SEQ ID NO: 64 - DNA, 27 bp, Artificial sequence
  Synthetic
atattaatactaccatcatcagttttg

SEQ ID NO: 65 - DNA, 28 bp, Artificial sequence
  Synthetic
tcaaaactgatgatggtagtattaatat

SEQ ID NO: 66 - DNA, 26 bp, Artificial sequence
  Synthetic
caatagacagacgtactaaagaatac

SEQ ID NO: 67 - DNA, 26 bp, Artificial sequence
  Synthetic
gtattctttagtagctctgtctattg

SEQ ID NO: 68 - DNA, 19 bp, Artificial sequence
  Synthetic
gattataggcacgtgccac

SEQ ID NO: 69 - DNA, 19 bp, Artificial sequence
  Synthetic
gtggcacgtgcctataatc

SEQ ID NO: 70 - DNA, 20 bp, Artificial sequence
  Synthetic
atctgtacatttgctgcctc

SEQ ID NO: 71 - DNA, 20 bp, Artificial sequence
  Synthetic
gaggcagcaaatgtacagat

SEQ ID NO: 72 - DNA, 25 bp, Artificial sequence
  Synthetic
tctacgatattagagctcaaattgt

SEQ ID NO: 73 - DNA, 25 bp, Artificial sequence
  Synthetic
gacaatttgagctctaatatcgtag

SEQ ID NO: 74 - DNA, 25 bp, Artificial sequence
  Synthetic
gcaaaatcatggtattctaataatt

SEQ ID NO: 75 - DNA, 25 bp, Artificial sequence
  Synthetic
tctccttatccacttatgaactaat

SEQ ID NO: 76 - DNA, 25 bp, Artificial sequence
  Synthetic
atgtttctctgtcctctgtattgac

SEQ ID NO: 77 - DNA, 25 bp, Artificial sequence
  Synthetic
ctgtcaatacagaggacagagaaac

SEQ ID NO: 78 - DNA, 25 bp, Artificial sequence
  Synthetic
taacctgactttcatgtagtcactt

SEQ ID NO: 79 - DNA, 21 bp, Artificial sequence
  Synthetic
acatgaaagtcaggttagtgg

SEQ ID NO: 80 - DNA, 23 bp, Artificial sequence
  Synthetic
aaaaaaactgtttactgcaggtt

SEQ ID NO: 81 - DNA, 23 bp, Artificial sequence
  Synthetic
aacctgcagtaaacagttttttt

SEQ ID NO: 82 - DNA, 25 bp, Artificial sequence
  Synthetic
ttgtgtgtgttaagaatctctcatt

SEQ ID NO: 83 - DNA, 25 bp, Artificial sequence
  Synthetic
atgagagattcttaacacacacaaa

SEQ ID NO: 84 - DNA, 25 bp, Artificial sequence
  Synthetic
gaaggttgtctatgtagtttttttc

SEQ ID NO: 85 - DNA, 21 bp, Artificial sequence
  Synthetic
tccagcactaaaactttagcc

SEQ ID NO: 86 - DNA, 18 bp, Artificial sequence
  Synthetic
aatccgcagccaaaccta

SEQ ID NO: 87 - DNA, 18 bp, Artificial sequence
  Synthetic
taggtttggctgcggatt

SEQ ID NO: 88 - DNA, 13 bp, Artificial sequence
  Synthetic
cgcgaggcgcgtc

SEQ ID NO: 89 - DNA, 42 bp, Artificial sequence
  Synthetic
ccggtcgtctggcaaggtggtatatctcgagatataccacct

SEQ ID NO: 90 - DNA, 12 bp, Artificial sequence
  Synthetic
gacgattttttg

SEQ ID NO: 91 - DNA, 40 bp, Artificial sequence
  Synthetic
ccgggcaatttcatagacctaattactcgagtaattaggt

SEQ ID NO: 92 - DNA, 14 bp, Artificial sequence
  Synthetic
aaattgcttttttg

SEQ ID NO: 93 - DNA, 39 bp, Artificial sequence
  Synthetic
ccggatctacgagtggatggtcaactcgagttgaccatc

SEQ ID NO: 94 - DNA, 13 bp, Artificial sequence
  Synthetic
gtagatctttttg

SEQ ID NO: 95 - DNA, 39 bp, Artificial sequence
  Synthetic
ccgggccggagtttagccagtccaactcgagttggactg

SEQ ID NO: 96 - DNA, 15 bp, Artificial sequence
  Synthetic
aactccggctttttg

SEQ ID NO: 97 - DNA, 39 bp, Artificial sequence
  Synthetic
ccggtgcttattggtattgtctcctctcgagaggagaca

SEQ ID NO: 98 - DNA, 14 bp, Artificial sequence
  Synthetic
aataagcatttttg

SEQ ID NO: 99 - DNA, 39 bp, Artificial sequence
  Synthetic
ccgggctggaatttactgggttcatctcgagatgaaccc

SEQ ID NO: 100 - DNA, 14 bp, Artificial sequence
  Synthetic
attccagctttttg

SEQ ID NO: 101 - DNA, 39 bp, Artificial sequence
  Synthetic
ccggctggaatttactgggttcattctcgagaatgaacc

SEQ ID NO: 102 - DNA, 14 bp, Artificial sequence
  Synthetic
aattccagtttttg

SEQ ID NO: 103 - DNA, 39 bp, Artificial sequence
  Synthetic
ccggcccagtccaaactactcaaagctcgagctttgagt

SEQ ID NO: 104 - DNA, 14 bp, Artificial sequence
  Synthetic
ggactgggtttttg

SEQ ID NO: 105 - DNA, 39 bp, Artificial sequence
  Synthetic
ccggcggaggattgaaccagtataactcgagttatactg

SEQ ID NO: 106 - DNA, 14 bp, Artificial sequence
  Synthetic
atcctccgtttttg

SEQ ID NO: 107 - DNA, 39 bp, Artificial sequence
  Synthetic
ccgggcattaccgatgccatgatatctcgagatatcatg

SEQ ID NO: 108 - DNA, 14 bp, Artificial sequence
  Synthetic
ggtaatgctttttg

(19) United States
(12) Patent Application Publication
(10) Pub. No.: US 2024/0299354 A1
(43) Pub. Date: Sep. 12, 2024

(54) COMPOSITIONS AND METHODS FOR TREATING CANCER

(71) Applicant: The Regents of the University of Michigan, Ann Arbor, MI (US)

(72) Inventor: Weiping Zou, Ann Arbor, MI (US)

(21) Appl. No.: 18/561,838

(22) PCT Filed: May 19, 2022
(86) PCT No.: PCT/US2022/029961

§ 371 Date: Nov. 17, 2023

Related U.S. Application Data
(60) Provisional application No. 63/190,888, filed on May 20, 2021.

(51) Int. Cl. A61K 31/4045; A61K 31/12; A61K 39/395; A61P 35/00; G01N 33/574

(52) U.S. Cl. CPC A61K 31/4045; A61K 31/12; A61K 39/3955; A61P 35/00; G01N 33/57484

(57) ABSTRACT

Provided herein are compositions and methods for cancer therapy. In particular, provided herein are compositions and methods for targeting SLC6A14 in cancer therapy using an agent that inhibits one or more activities of SLC6A14. Also, provided herein are compositions and methods for harnessing tumor SLC6A14 and SLC6A14-directed stiffness to overcome hypoxia-induced immune resistance and sensitize subjects to immunotherapy.

### STATEMENT OF RELATED APPLICATIONS

This application claims priority to U.S. Provisional Patent Application No. 63/190,888, filed May 20, 2021, the entire contents of which are incorporated herein by reference for all purposes.

### FIELD

Provided herein are compositions and methods for cancer therapy. In particular, provided herein are compositions and methods for targeting SLC6A14 in cancer therapy.

### BACKGROUND

One in eight women in the US develops breast cancer, and deaths from breast cancer account for 17% of all cancer deaths in women in the US (Cauley, J. A. et al. Study of Osteoporotic Fractures Research Group. Annals of internal medicine 130, 270-277 (1999); Miller, B. A., et al. Important advances in oncology, 193-207 (1994); Parker, S. L., et al. 1997. CA: a cancer journal for clinicians 47, 5-27 (1997)). Many breast cancers express PD1 and/or related ligands, indicating that these types of cancers may be suitable for checkpoint inhibitor therapies. However, these cancers respond with varying efficacy to checkpoint inhibition; and many patients also experience severe autoimmune related adverse advents to such therapy (Postow, M. A., et al. N Engl J Med 378, 158-168, (2018)).

The success of checkpoint inhibitor blockade as immunotherapy for some cancers is transforming the understanding of the interactions between cells of the immune system and malignant neoplasms. Limitations of checkpoint inhibitor treatment include the precipitation of a variety of autoimmune syndromes, which can be life-threatening, and the resistance of some cancer types, such as breast cancer, to this form of immunotherapy.

Improved immunotherapy for breast cancer is needed.

### SUMMARY

Provided herein are compositions and methods for cancer therapy. In particular, provided herein are compositions and methods for targeting SLC6A14 in cancer therapy.

Cytotoxic T cells (CTLs) exert mechanical force to potentiate target cell killing. Hypoxia is a hallmark of cancer. Experiments described herein found hypoxia diminished tumor sensitivity to CTL-killing. Hypoxia enhanced SLC6A14 and elevated tumor intracellular glutamine, resulting in Akt1 activation—thereby reducing FOXO1 and FOXO1-directed MYH9 and MYLK expression. Consequently, this weakened myosin II-mediated cell tension, impaired tumor membrane pore formation drilled by CTL-derived perform, and enabled tumor resistance to CTL-killing. Pathologically, high SLC6A14 expression correlated to enhanced tumor hypoxic density and reduced stiffness signature, and was associated with poor breast cancer patient survival. Tumor stiffness proteins positively correlated with checkpoint response rate in patients with cancer. Genetic and pharmacologic targeting cancer SLC6A14 or stiffness augmented tumor immunity and PD-L1 blockade efficacy. Thus, SLC6A14 functions as a metabolic and mechanical shared checkpoint. Accordingly, provided herein are compositions and methods for harnessing tumor SLC6A14 and SLC6A14-directed stiffness to overcome hypoxia-induced immune resistance and sensitize subjects to immunotherapy.

For example, in some embodiments, provided herein is a method of treating cancer, comprising: administering to a subject diagnosed with or suspected of having cancer an agent that inhibits one or more activities of SLC6A14.

Additional embodiments provide a method of treating cancer, comprising: administering to a subject an agent that inhibits one or more activities of SLC6A14 in combination with immunotherapy.

Other embodiments provide a method of treating cancer, comprising: a) assaying a sample from a subject diagnosed with or suspected of having cancer for the level of expression of SLC6A14; b) identifying the subject as having increased levels of expression of SLC6A14 relative to a control level; and b) administering an agent that inhibits one or more activities of SLC6A14 to the subject.

Further embodiments provide the use of an agent that inhibits one or more activities of SLC6A14 to treat cancer in a subject or an agent that inhibits one or more activities of SLC6A14 for use to treat cancer in a subject.

The present disclosure is not limited to a particular agent. Examples include, but are not limited to, an antibody (e.g., monoclonal antibody), a nucleic acid (e.g., a shRNA, an miRNA, and an antisense RNA), or a small molecule (e.g., α-MT). In some embodiments, the antibody is humanized. In some embodiments, the antibody is an antibody fragment (e.g., including but not limited to, Fab, Fab′, Fab′-SH, F(ab′)₂, Fv, or scFv). In some embodiments, the antibody or antibody fragment is humanized.

The present disclosure is not limited a particular cancer. In some embodiments, the cancer or cancer cells lack DNA repair activity. Examples of cancers include, but are not limited to, breast, lung, bladder, cervical, colon, head and neck, Hodgkin lymphoma, liver, renal cell, skin, stomach, and rectal.

In some embodiments, the method further comprises administering a second cancer therapy to the subject. Examples include, but are not limited to, chemotherapy and/or immunotherapy. Examples of immunotherapy include, but are not limited to, CAR-T therapy, TCR therapy, antibody immunotherapy, or checkpoint inhibitors. In some embodiments, the immune checkpoint inhibitor is ipilimumab, nivolumab, pembrolizumab, or atezolizumab.

Additional embodiments are described herein.

### DESCRIPTION OF THE FIGURES

FIG. 1. Hypoxia endows tumor resistance to CTL-killing via SLC6A14-mediated amino acid uptake a, Effect of hypoxia on CTL-tumor killing. n=3 biological replicates; ** P=0.0077 (two-tailed t-test). b, Effect of hypoxia on perforin and granzyme B-tumor killing. n=3 biological replicates; **** P<0.0001 (4T1), * P=0.0151 (PY8119), * P=0.0105 (MDA-MB231), * P=0.0126 (T47D) (two-tailed t-test). c, Effect of low amino acid levels on tumor cell death. d, Effect of low amino acid levels on CTL-tumor killing. n=3 biological replicates; ** P<0.01; *** P<0.001 (two-tailed t-test). e, Scheme shows SLCs are capable of transporting more than 3 amino acids among Asn, Arg, Gln, Gly, and Ser. f, Role of hypoxia in SLCs. 4T1 cells were exposed to hypoxia. g-h, Role of hypoxia in SLC6A14 stability. Immunoblots showed HIF1α and SLC6A14 (g). SLC6A14 protein half-life is shown (h). i, Representative images show pimonidazole (PIMO) and SLC6A14 staining in 4T1 tumors. Scale bar, 500 μm. n=5. j, Representative images show SLC6A14 and CAIX staining in human basal-like breast cancer tissues. Scale bar, 50 μm. n=85. k, Correlation between SLC6A14 and CAIX in human basal-like breast cancer tissues. Each data point represents the value from the individual patient. r=0.4666, P<0.0001, n=85 (Pearson's correlation test). 1-m, Representative mass spectrometry images show in vivo tumor glutamine in mice bearing Slc6a14^{+/+} (n=6) or Slc6a14^{-/-} (n=5) 4T1 tumors. Scale bar, 4 mm. (1). Results are shown as the average intensity of glutamine signal per pixel. ** P=0.0047 (two-tailed t-test) (m). n, Intracellular concentration of glutamine in Scl6a14^{+/+} and Scl6a14^{-/-} 4T1 cells. n=3 biological replicates. ** P=0.0011 (two-tailed t-test).

FIG. 2. SLC6A14 diminishes tumor receptivity to CTL-killing via altering membrane pores a, Effect of SLC6A14 on perform and granzyme B-mediated tumor cell death. n=3 biological replicates; *** P=0.0002, *** P=0.0003 (two-tailed t-test). b, Effect of hypoxia on perform and granzyme B-mediated tumor cell death. n=3 biological replicates; *** P=0.0002, * P=0.0014, N.S, P>0.05. (one-way ANOVA). c, Effect of Slc6a14 on CTL-tumor killing. n=3 biological replicates; **** P<0.0001 (two-tailed t-test). d-e, Effect of Slc6a14 on perforin-mediated membrane pore formation. Fluorescence microscope images showed PI⁺ (red) tumor cells. Scale bar, 30 μm (d). FACS showed percentage of PI⁺ tumor cells (e), n=3 biological replicates; *** P=0.0001 (two-tailed t-test). f-g, Effect of Slc6a14 on CTL-mediated membrane pore formation. Fluorescence microscope images showed PI+ (yellow) and PI− (green) cells (f). Percentage of PI+ cells was shown in (g). 500 cells per field, 3 fields per group. Scale bar=100 μm; *** P=0.0005 (two-tailed t-test). h-k, Effect of Slc6a14 on perforin-mediated membrane pore formation. * P<0.05, ** P<0.01, *** P<0.001 (two-tailed t-test).

FIG. 3. Hypoxia reduces cancer stiffness by targeting myosin II via SLC6A14 a, Illustration of the Optical Tweezers Microscope (OTM) method capable of making local and short time-scale mechanical measurements on living cells. b, Effect of hypoxia on 4T1 cell stiffness. 4T1 cells were exposed to normoxia and hypoxia. Total cell counts: n=15/group, **** P<0.0001 (two-tailed t-test). c, Effect of hypoxia on breast cancer cell F-actin. Scale bar, 20 μm. d, Effect of Slc6a14 on hypoxia-directed 4T1 cell stiffness. n=40/group, **** P<0.0001 (one-way ANOVA). e, Effect of hypoxia on MCF7 cell stiffness gene signature. Total cell counts: n=40/group, **** P<0.0001 (two-tailed t-test). g, h, Effect of hypoxia on Myh9 and Mylk transcripts (g) and proteins (h) in 4T1 cells. Myh9 and Mylk transcripts (g) and proteins (h) were measured by real-time PCR (g) and immunoblots (h). n=3 biological replicates; **** P<0.0001, ** P=0.0037, (two-tailed t-test). I, j, Effect of hypoxia on MYH9 and MYLK transcripts (i) and proteins (j) in MCF7 cells. MYH9 and MYLK transcripts (i) and proteins (j) were measured by real-time PCR (i) and immunoblots (j). n=3 biological replicates; * P=0.0374, *** P<0.0001, (two-tailed t-test). k, 1, Effect of Slc6a14 on Myh9 and Mylk transcripts (k) and proteins (1) in 4T1 cells. Myh9 and Mylk transcripts (k) and proteins (1) were measured by real-time PCR (k) and immunoblots (l) in SLC6A14^{+/+} and SLC6A14^{-/-} 4T1 cells. n=3 biological replicates; *** P=0.0010 (Myh9), **** P<0.0001 (Mylk), (two-tailed t-test). m, Effect of SLC6A14 on human breast cancer stiffness gene signature.

FIG. 4. SLC6A14 alters FOXO1 expression to control tumor stiffness genes a, MYH9 and MYLK transcription factors (TFs) screening. b-c, ChIP showed the enrichment of FOXO1 at the MYH9 (b) or MYLK (c) promoters in MDA-MB231 cells. n=4; ** P<0.01, **** P<0.0001, (two-tailed t-test). d, JASPAR predicted FOXO1 binding sequence in the MYH9 (pMYH9-1, -3 and -13 regions) or MYLK (pMYLK-2 region) promoters. e, f, Effect of FOXO1 on MYH9 and MYLK transcripts (e) and proteins (f) in T47D cells. MYH9 and MYLK transcripts (e) and (f) were detected by PCR (e) and immune blots (f) in T47D cells expressing scrambled shRNA or two sh-FOXO1 (sh-FOXO1 #1, #2). n=3 biological replicates, ** P<0.01, *** P<0.001 (two-tailed t-test). g, Effect of FOXO1 on T47D cell stiffness. Total cell counts: n=18 (Scramble), n=14 (sh-FOXO1), **** P<0.0001 (two-tailed t-test). h, Effects of FOXO1 and 4-HAP on perform and granzyme B-mediated T47D cell death. n=3 biological replicates, **** P<0.0001, ** P=0.0044, * P=0.0163 (one-way ANOVA). i, Effect of hypoxia on Foxo1 expression in 4T1 cells. j, Effect of Slc6a14 on Foxo1 expression in 4T1 cells. k, Effect of FOXO1 on stiffness protein expression in T47D cells. 1, Effect of low glutamine on stiffness-related proteins in MDA-MB231 cells. m, Role of the molecular cascade of SLC6A14, Akt, and FOXO1 in cancer stiffness proteins in the context of hypoxia.

FIG. 5. Targeting tumor SLC6A14 and stiffness affect tumor immunity and immunotherapy a, Scl6a14^{+/+} and Scl6a14^{-/-} 4T1 tumor growth in wild type BALB/c mice. n=10 per group. **** P<0.0001 (two-way ANOVA). b, Cleaved caspase-3 (CC3) staining in Slc6a14^{+/+} and Slc6a14^{-/-} 4T1 tumor tissues (n=5 mice/group). Scale bar, 25 μm. **P=0.0014 (two-tailed t-test). c, Effect of CD8⁺ T cells on Slc6a14^{-/-} 4T1 tumor growth. **P=0.0031 on day 36 (two-way ANOVA). d-f, Effect of α-MT on tumor stiffness gene expression (d, e) and stiffness (f). Myh9 and Mylk transcripts (d) and proteins (e) were detected by real-time PCR (d) and immunoblots (e) in 4T1 tumors. n=5/group, ***P=0.0006, *P=0.0196 (two-tailed t-test). Results are expressed as log 10 (pascal). n=4, ****P<0.0001 (two-way ANOVA) (f). g-h, Correlation between stiffness proteins and checkpoint therapy response. Proteomic analysis quantified MYH9 (g) and MYLK (h) proteins in tumor biopsies from patients with melanoma treated with anti-PD-1. The clinical response rates are shown in melanoma patients with high and low levels of MYH9 (g) and MYLK (h). Complete response (CR), n=10; partial response (PR), n=30; progressive disease (PD), n=27. Chi-square test, ***P<0.001; ****P<0.0001. i-j, Effect of α-MT in combination with PD-L1 blockade on tumor growth and immune responses, (n=6/group). Tumor volumes (i) and tumor weight (j) are shown. *P<0.05, **P<0.01; two-way ANOVA for (i) and one-way ANOVA for (j). k-r, Effect of 4-HAP in combination with PD-L1 blockade on tumor growth and immune responses, (n=6/group). Tumor weight (k) and tumor volumes (1) are shown. The percentage of tumor infiltrating granzyme-B⁺ CD8⁺ T cells (m, n), and tumor training lymph node (TDLN) TNF-α⁺ (o, p) and IFN-γ⁺ (q, r) *P<0.05, **P<0.01, ***P<0.001, ****P<0.0001; two-way ANOVA for (1), or one-way ANOVA for (k, m-r).

FIG. 6. Hypoxia endows tumor resistance to CTL-killing via SLC6A14-mediated amino acid uptake. a, Effect of hypoxia on MHC-I-mediated antigen presentation. n=3 biological replicates, P>0.05. b, Effect of low amino acid levels on tumor cell death. n=3 biological replicates; * P<0.05, ** P<0.01, *** P<0.001 (two-tailed t-test). c-e, Role of hypoxia in SLCs. Immunoblots showed SLC expression in MDA-MB231 (c), T47D (d), and PY8119 (e) cells. n=3 biological replicates. f, Role of hypoxia in SLC6A14 transcripts. g, Slc6a14 knockout efficiency. n=3 biological replicates. h, Schematic diagram showed 4T1 tumor-bearing model for in vivo amino acid detection with AFADESI-MSI. i-j, Representative mass spectrometry images showed in vivo tumor serine, asparagine, and arginine in mice bearing Slc6a14^{+/+} (n=6) or Slc6a14^{-/-} (n=5) 4T1 tumors. Scale bar, 4 mm. (i). Results are shown as the average intensity of amino acid signal per pixel. Data plotted are mean±s.e.m; P>0.05 (two-tailed t-test) (j).

FIG. 7. SLC6A14 diminishes tumor receptivity to CTL-killing via altering membrane pores. a, Effect of hypoxia and low glutamine on perform and granzyme B-mediated tumor cell death. n=3 biological replicates, P>0.05 (one-way ANOVA). b, Knocking down efficiency of sh-Slc6a14. c, Effect of SLC6A14 on MHC-I-mediated antigen presentation. n=3 biological replicates. P>0.05 (one-way ANOVA).

FIG. 8. Hypoxia reduces cancer stiffness by targeting myosin II via SLC6A14. a, Effect of hypoxia on PY8119 cell stiffness. Total cell counts: n=15/group, **** P<0.0001 (two-tailed t-test). b, Effect of Slc6a14 on 4T1 cell stiffness. n=38-40/group, **** P<0.0001 (two-tailed t-test). c, Effect of α-MT on tumor stiffness. Total cell counts: n=14/group, ** P=0.0017 (two-tailed t-test). d-e, Effect of hypoxia on stiffness related gene transcripts in 4T1 (d) and MCF7 (e) cells. n=3 biological replicates, P>0.05 (two-tailed t-test). f, Effect of hypoxia on Myh9 and Mylk proteins in Slc6a14^{-/-} 4T1 cells.

FIG. 9. SLC6A14 alters FOXO1 expression to control tumor stiffness genes. a-b, Schematic diagram showing putative FOXO1 binding regions on the promoters of MYH9 (a) and MYLK (b). c, Effect of Foxo1 on tumor stiffness gene expression. n=3 biological replicates. ** P<0.01, *** P<0.001. Data plotted are mean±s.e.m. d, Schematic diagram illustrates the molecular cascade by which hypoxia endows tumor resistance to CTL-killing.

FIG. 10. Targeting tumor SLC6A14 and stiffness affects tumor immunity and immunotherapy. a-b, Scl6a14^{+/+} and Scl6a14^{-/-} 4T1 tumor masses (a) and weight (b) are shown. Scale bar, 1 cm. n=10 per group. **** P<0.0001 (two-tailed t-test). c, Effect of SLC6A14 on tumor-bearing mice survival. n=10/group, P<0.0001 (log-rank test). d, Relationship between SLC6A14 expression and breast cancer patient survival. Low SLC6A14, n=39; High SLC6A14, n=48, P=0.0209 (log-rank test). e, Ki-67 staining in Slc6a14^{+/+} and Slc6a14^{-/-} 4T1 tumors in vivo. n=3 mice/group, Scale bar, 25 μm. Results are shown as percentage of Ki-67⁺ cells per view. P>0.05 (two-tailed t-test). f-i, Effect of 4-HAP in combination with PD-L1 blockade on tumor infiltrating T cells. The percentage of IFNγ⁺ (f, g) and TNF-α⁺ (h, i) CD8⁺ T cells in tumor tissues. n=6/group, * P=0.0389 (f); * P=0.0134 (h) (one-way ANOVA).

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject diagnosed with cancer” refers to a subject who has been tested and found to have cancer. As used herein, the term “initial diagnosis” refers to a test result of initial disease that reveals the presence or absence of disease.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of an agent (e.g., an agent described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., an agent described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent or agents with a carrier, inert or active, making the composition especially suitable for diagnostic or therapeutic use in vivo, or ex vivo.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries.

As used herein “immunoglobulin” refers to any class of structurally related proteins in the serum and the cells of the immune system that function as antibodies. In some embodiments, an immunoglobulin is the distinct antibody molecule secreted by a clonal line of B cells.

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “anti-SLC6A14 antibody” refers to an antibody which specifically recognizes an antigen and/or epitope presented by SLC6A14.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods; See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab′” fragment comprises one light chain and the C_{H1} and variable region of one heavy chain. The heavy chain of a Fab molecule cannot form a disulfide bond with another heavy chain molecule. A “Fab” fragment comprises one light chain and one heavy chain that comprises an additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

As used herein, the term “chimeric antibody” refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

As used herein, the term “natural antibody” refers to an antibody in which the heavy and light chains of the antibody have been made and paired by the immune system of a multicellular organism. For example, the antibodies produced by the antibody-producing cells isolated from a first animal immunized with an antigen are natural antibodies. Natural antibodies contain naturally-paired heavy and light chains. The term “natural human antibody” refers to an antibody in which the heavy and light chains of the antibody have been made and paired by the immune system of a human subject.

Native human light chains are typically classified as kappa and lambda light chains. Native human heavy chains are typically classified as mu, delta, gamma, alpha, or epsilon, and define the antibody's isotype as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids. See, e.g., Fundamental Immunology (1989) Ch. 7 (Paul, W., ed., 2nd ed. Raven Press, N.Y.); herein incorporated by reference in its entirety.

The term “antigen-binding site” refers to a portion of an antibody capable of specifically binding an antigen. In certain embodiments, an antigen-binding site is provided by one or more antibody variable regions.

The term “epitope” refers to any polypeptide determinant capable of specifically binding to an immunoglobulin or a T-cell or B-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics.

As used herein, the term “multivalent”, particularly when used in describing an agent that is an antibody, antibody fragment, or other binding agent, refers to the presence of two or more (e.g., 2, 3, 4, 5, 6, 7, 8, 9, 10, or more) antigen binding sites on the agent.

As used herein, the term “multispecific,” particularly when used in describing an agent that is an antibody, antibody fragment, or other binding agent, refers to the capacity to of the agent to bind two or more (e.g., 2, 3, 4, 5, 6, 7, 8, 9, 10, or more) targets (e.g., unrelated targets). For example, a bispecific antibody recognizes and binds to two different antigens.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class; whereas conservative substitutions may involve the exchange of a member of one of these classes for another member of that same class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “selectively” (e.g., as in “selectively targets,” “selectively binds,” etc.) refers to the preferential association of an agent (e.g., antibody or antibody fragment) for a particular entity (e.g., antigen, antigen presenting cell, etc.). For example, an agent selectively targets a particular cell population if it preferentially associates (e.g., binds an epitope or set of epitopes presented thereon) with that cell population over another cell population (e.g., all other cell populations present in a sample). The preferential association may be by a factor of at least 2, 4, 6, 8, 10, 20, 50, 100, 10¹, 10⁴, 10⁵, 10⁶, or more, or ranges there between. An agent that X-fold selectively targets a particular cell population, associates with that cell population by at least X-fold more than other cell populations present.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include, but are not limited to, single-stranded and double-stranded compounds, such as, antisense oligonucleotides, siRNAs and shRNAs.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases. “Bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

“Oligonucleotide” means a polymer of linked nucleosides each of which can be modified or unmodified, independent one from another.

### DETAILED DESCRIPTION OF THE DISCLOSURE

Provided herein are compositions and methods for cancer therapy and for identifying and testing cancer therapies. In particular, provided herein are compositions and methods for targeting SLC6A14 in cancer therapy.

Hypoxia is a common feature of most solid tumors. Tumor adaptation to hypoxia includes several layers of coordinated events, including metabolic reprogramming (such as increased aerobic glycolytic and amino acid metabolism) —thereby supporting tumor cell proliferation, survival, and metastasis¹. In addition to the most well-known “Warburg effect” featured as activated aerobic glycolysis, recent studies have pointed toward the importance of hypoxia-induced active glutamine metabolism, an additional biological characteristic of most cancer cells^{2,3}. However, a link between hypoxia-directed glutamine metabolism and cancer immune responses, particularly CD8⁺ cytotoxic T cell (CTL) activity, has remained unidentified.

Stiffness is an intrinsic feature of a cell. Cancer cells with a soft membrane may be easily deformable, then invasively migrate to the surrounding tissues⁴. Cancer progenitor cells (or stem-like cells) appear to be softer than their counterparts^{5,6}. This may provide a mechanism by which cancer stemness is associated with chemotherapy resistance and poor patient outcome⁷⁻⁹.

Immunotherapy has revolutionized cancer treatment, yet most patients do not respond^{10,11}. Accumulating evidence has shed light on immunotherapy resistance mechanisms—including the involvement of immunosuppressive networks, cancer genetic mutation burdens, genetic and epigenetic mutations in MHC- and IFN-signaling pathways, impaired effector T cell tumor trafficking, and abnormal metabolic impact on T cells¹²⁻²². In this context, hypoxia may promote the development and expansion of myeloid derived suppressor cells and Foxp3⁺ regulatory T cells to enable tumor resistance to checkpoint blockade^{23,24}. Nonetheless, CD8⁺ T cells are the major effector cells in spontaneous and immunotherapy-induced anti-tumor immunity in the immune system^{25,26}. Perforin and granzymes are stored in lytic granules in activated CD8⁺ cytotoxic T cells. CTL-derived perform drills membrane pores in tumor cells, killing tumor cells via granzyme B and other mediators^{27,28}. Perform-mediated pore formation is the first step for CTL-initiated tumor killing. CTLs use mechanical force to potentiate target cell killing²⁹.

Experiments described herein demonstrated that hypoxia enhanced expression of SLC6A14 (an amino acid transporter) and elevated tumor intracellular glutamine, causing Akt1 activation—resulting in reduction of FOXO1 (forkhead box protein O1) and FOXO1-directed MYH9 (Myosin heavy chain 9) and MYLK (myosin light chain kinase) expression. Consequently, this weakened myosin II-mediated cell tension and membrane stiffness, impaired tumor membrane pore formation drilled by CTL-derived perforin, and reduced tumor cell sensitivity to CTL-killing.

Accordingly, in some embodiments the present disclosure provides compositions and methods for treating cancer by blocking SLC6A14 activity (e.g., in combination with immunotherapy). In some embodiments, the present disclosure provides compositions and methods for blocking SLC6A14 activity in cancer cells and testing the efficacy and/or safety of therapeutic agents or modalities (e.g., immunotherapies) against the cells having reduced SLC6A14 activity.

### I. Inhibitors

Provided herein are inhibitors of SLC6A14 activity (e.g., signaling, interaction with its ligands, expression, etc.). In some embodiments, inhibitors interact directly with SLC6A14 (e.g., by physically interacting with an SLC6A14 polypeptide or nucleic acid encoding a SLC6A14 polypeptide). In other embodiments, inhibitors interact or block an activity of a component of a SLC6A14 pathway (e.g., upstream or downstream signaling partners of SLC6A14).

In some embodiments, the inhibitor is selected from, for example, a small molecule, a nucleic acid, a peptide, an aptamer, or an antibody or antibody-like molecule. In some embodiments, the antibody is an anti-SLC6A14 antibody.

### A. Antibodies

Any suitable antibody (e.g., monoclonal, polyclonal, or synthetic) may be utilized in the methods disclosed herein. In some embodiments, commercially available antibodies to SLC6A14 are utilized (e.g., available from Abcam, Cambridge, United Kingdom; Thermo Fisher, Waltham, MA; Sigma Aldrich, St. Louis, MO, etc.)

In some embodiments, the antibodies are humanized antibodies. Methods for humanizing antibodies are described (See e.g., U.S. Pat. Nos. 6,180,370, 5,585,089, 6,054,297, and 5,565,332; each of which is herein incorporated by reference).

In some embodiments, the immunoglobulin molecule is composed of two identical heavy and two identical light polypeptide chains, held together by interchain disulfide bonds. Each individual light and heavy chain folds into regions of about 110 amino acids, assuming a conserved three-dimensional conformation. The light chain comprises one variable region (termed VL) and one constant region (CL), while the heavy chain comprises one variable region (VH) and three constant regions (CH1, CH2 and CH3). Pairs of regions associate to form discrete structures. In particular, the light and heavy chain variable regions, VL and VH, associate to form an “FV” area that contains the antigen-binding site.

The variable regions of both heavy and light chains show considerable variability in structure and amino acid composition from one antibody molecule to another, whereas the constant regions show little variability. Each antibody recognizes and binds an antigen through the binding site defined by the association of the heavy and light chain, variable regions into an FV area. The light-chain variable region VL and the heavy-chain variable region VH of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDR's) are found in each of the light and heavy chains. The three CDRs from a light chain and the three CDRs from a corresponding heavy chain form the antigen-binding site.

The amino acid sequences of many immunoglobulin heavy and light chains have been determined and reveal two important features of antibody molecules. First, each chain consists of a series of similar, although not identical, sequences, each about 110 amino acids long. Each of these repeats corresponds to a discrete, compactly folded region of protein structure known as a protein domain. The light chain is made up of two such immunoglobulin domains, whereas the heavy chain of the IgG antibody contains four.

The second important feature revealed by comparisons of amino acid sequences is that the amino-terminal sequences of both the heavy and light chains vary greatly between different antibodies. The variability in sequence is limited to approximately the first 110 amino acids, corresponding to the first domain, whereas the remaining domains are constant between immunoglobulin chains of the same isotype. The amino-terminal variable or V domains of the heavy and light chains (V_{H} and V_{L}, respectively) together make up the V region of the antibody and confer on it the ability to bind specific antigen, while the constant domains (C domains) of the heavy and light chains (C_{H} and C_{L}, respectively) make up the C region. The multiple heavy-chain C domains are numbered from the amino-terminal end to the carboxy terminus, for example C_{H}1, C_{H}2, and so on.

The protein domains described above associate to form larger globular domains. Thus, when fully folded and assembled, an antibody molecule comprises three relatively equal-sized globular portions joined by a flexible stretch of polypeptide chain known as the hinge region. Each arm of this Y-shaped structure is formed by the association of a light chain with the amino-terminal half of a heavy chain, whereas the trunk of the Y is formed by the pairing of the carboxy-terminal halves of the two heavy chains. The association of the heavy and light chains is such that the V_{H} and V_{L} domains are paired, as are the C_{H}1 and C_{L} domains. The C_{H}3 domains pair with each other but the C_{H}2 domains do not interact; carbohydrate side chains attached to the C_{H}2 domains lie between the two heavy chains. The two antigen-binding sites are formed by the paired V_{H} and V_{L} domains at the ends of the two arms of the Y.

Proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments are identical and contain the antigen-binding activity. These are termed the Fab fragments, for Fragment antigen binding. The Fab fragments correspond to the two identical arms of the antibody molecule, which contain the complete light chains paired with the V_{H} and C_{H}1 domains of the heavy chains. The other fragment contains no antigen-binding activity but was originally observed to crystallize readily, and for this reason was named the Fc fragment, for Fragment crystallizable. This fragment corresponds to the paired C_{H}2 and C_{H}3 domains and is the part of the antibody molecule that interacts with effector molecules and cells. The functional differences between heavy-chain isotypes lie mainly in the Fc fragment. The hinge region that links the Fc and Fab portions of the antibody molecule is in reality a flexible tether, allowing independent movement of the two Fab arms, rather than a rigid hinge.

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

In some embodiments, the antibody is a chimeric antibody (e.g., comprising a variable region or CDR sequences described herein and a different constant region). In some embodiments, chimeras comprise constant region sequences from a different species or isotype as described herein. In some embodiments, the antibody is a fragment (e.g., a fragment that retains binding to SLC6A14 or other target).

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

The disclosure also features methods for producing any of the antibodies or antigen-binding fragments thereof described herein. In some embodiments, methods for preparing an antibody described herein can include immunizing a subject (e.g., a non-human mammal) with an appropriate immunogen. For example, to generate an antibody that binds to SLC6A14, one can immunize a suitable subject (e.g., a non-human mammal such as a rat, a mouse, a gerbil, a hamster, a dog, a cat, a pig, a goat, a horse, or a non-human primate) with a full-length or fragment of a SLC6A14 polypeptide.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a SLC6A14 polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybridoma cells are then screened for secretion of the desired antibodies, e.g., an antibody that binds to SLC6A14.

The antibodies or antigen-binding fragments thereof described herein can be produced using a variety of techniques in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof are expressed in, and purified from, transgenic animals (e.g., transgenic mammals). For example, an antibody is produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3.sup.rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are include e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### B. Nucleic Acids

In some embodiments, the SLC6A14 inhibitor is a nucleic acid. Exemplary nucleic acids suitable for inhibiting SLC6A14 (e.g., by preventing expression of SLC6A14) include, but are not limited to, antisense nucleic acids and RNAi. In some embodiments, nucleic acid therapies are complementary to and hybridize to at least a portion (e.g., at least 5, 8, 10, 11, 12, 13, 14, 15, 16, 17, 18, 19, or 20 nucleotides) of SLC6A14.

In some embodiments, compositions comprising oligomeric antisense compounds, particularly oligonucleotides are used to modulate the function of nucleic acid molecules encoding SLC6A14, ultimately modulating the amount of SLC6A14 expressed. This is accomplished by providing antisense compounds that specifically hybridize with one or more nucleic acids encoding SLC6A14. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is decreasing the amount of SLC6A14 proteins in the cell.

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases. Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the RISC pathway and include, without limitation, siRNA, ssRNA and microRNA mechanisms; and occupancy based mechanisms. Certain antisense compounds may act through more than one such mechanism and/or through additional mechanisms.

In certain embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region.

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO/2011/139702.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

Additional modifications are described, for example, in U.S. Pat. No. 9,796,976, herein incorporated by reference in its entirety.

In some embodiments, nucleic acids are RNAi nucleic acids. “RNA interference (RNAi)” is the process of sequence-specific, post-transcriptional gene silencing initiated by a small interfering RNA (siRNA), shRNA, or microRNA (miRNA). During RNAi, the RNA induces degradation of target mRNA with consequent sequence-specific inhibition of gene expression.

In “RNA interference,” or “RNAi,” a “small interfering RNA” or “short interfering RNA” or “siRNA” or “short hairpin RNA” or “shRNA” molecule, or “miRNA” an RNAi (e.g., single strand, duplex, or hairpin) of nucleotides is targeted to a nucleic acid sequence of interest, for example, SLC6A14.

An “RNA duplex” refers to the structure formed by the complementary pairing between two regions of a RNA molecule. The RNA using in RNAi is “targeted” to a gene in that the nucleotide sequence of the duplex portion of the RNAi is complementary to a nucleotide sequence of the targeted gene. In certain embodiments, the RNAi is are targeted to the sequence encoding SLC6A14. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure (e.g., shRNA). In addition to the duplex portion, the hairpin structure may contain a loop portion positioned between the two sequences that form the duplex. The loop can vary in length. In some embodiments the loop is 5,6,7,8,9, 10, 11, 12, 13, 14, 15, 16, 17, 18, 19,20,21,22,23,24,25,26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter. The nucleic acid sequence can also include a polyadenylation signal. In some embodiments, the polyadenylation signal is a synthetic minimal polyad n certain embodiments, provided herein are compounds comprising a modified oligonucleotide consisting of 12 to 30 linked nucleosides and comprising a nucleobase sequence comprising a portion of at least 8, at least 10, at least 12, at least 14, at least 15, at least 16, at least 17, at least 18, at least 19, or at least 20 contiguous nucleobases complementary to an equal length portion of SLC6A14.

In some embodiments, hybridization occurs between an antisense compound disclosed herein and an SLC6A14 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as an SLC6A14 nucleic acid).

Non-complementary nucleobases between an antisense compound and an SLC6A14 nucleic acid may be tolerated provided that the antisense compound remains able to specifically hybridize to a target nucleic acid. Moreover, an antisense compound may hybridize over one or more segments of an SLC6A14 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to an SLC6A14 nucleic acid, a target region, target segment, or specified portion thereof. Percent complementarity of an antisense compound with a target nucleic acid can be determined using routine methods.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. In certain embodiments, the antisense compounds are complementary to at least an 18 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

The present disclosure contemplates the use of any genetic manipulation for use in modulating the expression of SLC6A14. Examples of genetic manipulation include, but are not limited to, gene knockout (e.g., removing the SLC6A14 gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present disclosure, vectors are administered into tumors or tissue associated with tumors using direct injection. In other embodiments, administration is via the blood or lymphatic circulation (See e.g., PCT publication 1999/02685 herein incorporated by reference in its entirety). Exemplary dose levels of adenoviral vector are preferably 10⁸ to 10¹¹ vector particles added to the perfusate.

In some embodiments, CRISPR/Cas9 systems or related systems are used to delete or knock out genes. Clustered regularly interspaced short palindromic repeats (CRISPR) are segments of prokaryotic DNA containing short, repetitive base sequences. These play a key role in a bacterial defense system, and form the basis of a genome editing technology known as CRISPR/Cas9 that allows permanent modification of genes within organisms.

### C. Additional Inhibitors

Additional inhibitors include, for example, small molecules. In some embodiments, the small molecule is α-Methyltryptamine (α-MT; Indopan) or 4′-Hydroxyacetophenone (4-HAP).

### D. Pharmaceutical Compositions

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intra-tracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may additionally contain other adjunct components conventionally found in pharmaceutical compositions. Thus, for example, the compositions may contain additional, compatible, pharmaceutically-active materials such as, for example, anti-pruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

### II. Treatment Methods

The compositions and methods described herein find use in the treatment of a variety of cancer types (e.g., including but not limited to, breast, lung, bladder, cervical, colon, head and neck, Hodgkin lymphoma, liver, renal cell, skin, stomach, and rectal, and other tumors or cancers).

In some embodiments, the level of expression of SLC6A14 is assayed before or during treatment with a SLC6A14 inhibitor. In some embodiments, subjects with increased levels of SLC6A14 are identified as candidates for treatment with an SLC6A14 inhibitor (e.g., alone or in combination with immunotherapy). In some embodiments, a level of SLC6A14 greater than a control level (e.g., the level in subjects not diagnosed with cancer or the level in subjects identified as not responding to immunotherapies) identifies as subject as a candidate for treatment with a SLC6A14 inhibitor.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual antibodies, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the antibodies are administered in maintenance doses, ranging from 0.01 μg to 100 g per kg of body weight, once or more daily, to once every 20 years.

Some embodiments of the present disclosure provide methods for administering an effective amount of a compound described herein and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies).

In some embodiments, the therapies described herein are used in combination with immunotherapies (e.g., CAR-T/TCR, antibody immunotherapy, and/or checkpoint inhibitors). See e.g., Roth S, et al., Ann Surg Oncol. 2018 October; 25(11):3404-3412. Epub 2018 Jul. 23; and U.S. Pat. Nos. 9,943,579, 9,937,247, 10,081,665, and 10,072,082; each of which is herein incorporated by reference in its entirety. In some embodiments, the immune checkpoint inhibitor is ipilimumab, nivolumab, pembrolizumab, or atezolizumab.

In a particular embodiment, the additional therapeutic agent(s) is an anticancer agent. A number of suitable anticancer agents are contemplated for use in the methods of the present disclosure. Indeed, the present disclosure contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteasome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TNF-related apoptosis-inducing ligand (TRAIL), antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethimide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present disclosure provide a compound of the disclosure and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide); and 17) gonadotropin-releasing hormone analogs (e.g., leuprolide).

Any oncolytic agent that is routinely used in a cancer therapy context finds use in the compositions and methods of the present disclosure. For example, the U.S. Food and Drug Administration maintains a formulary of oncolytic agents approved for use in the United States. International counterpart agencies to the U.S.F.D.A. maintain similar formularies. Table II provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflomithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafamib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpimase, rebeccamycin analogue, rhuAngiostatin protein, rhuMab 2C4, rosiglitazone, rubitecan, S-1, 5-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

### EXPERIMENTAL

The following examples are provided in order to demonstrate and further illustrate certain preferred embodiments and aspects of the present disclosure and are not to be construed as limiting the scope thereof.

### Example 1

### Methods

### Cell Culture

4T1, PY8119, T47D, MDA-MB231, and HEK 293T cells were obtained from the American Type Culture Collection (ATCC). MDA-MB231 and HEK 293T cells were cultured in DMEM medium (11995065, Gibco). 4T1 and T47D cells were cultured in RPMI 1640 medium (SH3025501, HyClone). PY8119 cells were cultured in F12K medium (21127022, Gibco). All cell lines were supplemented with 10% FBS (FB61, ASi) and 1% penicillin/streptomycin (Gibco), unless otherwise indicated. Cells were grown in a humidified incubator (Galaxy 170 s, New Brunswick) with 5% CO2 at 37° C. Cells were regularly tested and verified to be mycoplasma negative using MycoAlert™ Detection Kit (LT07-318, LONZA).

Cells were cultured in normoxia (21% O₂) and hypoxia (1% O₂) for 24 hours, unless otherwise indicated. For the low levels of individual amino acid culture (amino acid starvation): cells were cultured in RPMI1640 medium containing 10% dialyzed FBS (1841165, Gibco), 1% penicillin/streptomycin, and all original amino acid supplementation—excluding one indicated amino acid with 2% of the original concentration (Table 3).

### Genetic Knockout and Knockdown Cells

Slc6a14 knockout (Slc6A14^{-/-}) 4T1 cells were generated using CRISPR technology. Mouse Slc6a14 locus was targeted using Slc6a14 Double Nickase Plasmids (m) (sc-425301-NIC, Santa Cruz). Tumor cells were transfected with the plasmid using Lipofectamine 2000 (Thermo Fisher Scientific). Twenty-four hours after transfection, cells were selected with 10 μg/ml puromycin (Santa Cruz Biotechnology) for another 72 hours. Single cell clones were selected and expanded in 96-well plates. Control double nickase plasmids (sc-437281, Santa Cruz) were used as negative control. Knockout clones were identified by immunoblotting. Multiple clones were used for the study.

To establish cells stably expressing sh-SLC6A14, cells were infected with relevant shRNAs expressing lentiviral particles. Stable transfectants were selected in medium containing 10 μg/ml puromycin (A1113803, Gibco). After 2-3 passages in the presence of hygromycin, cells were used for experiments without cloning. shRNA bacterial glycerol (pLKO.1 puro) was obtained from the Sigma MISSION shRNA Bacterial Glycerol Stock. ShRNA sequences were packed into a lentivirus packaging construct and transfected into HEK293T cells with Lipofectamine™ 2000 transfection reagent (11668019, Invitrogen). The sequences of all shRNAs are listed in FIG. 11.

### Human Breast Cancer Tissue Microarray

Paraffin-embedded human breast tissue microarray from one cohort was used in this study. Formalin-fixed, paraffin-embedded breast cancer tissue blocks (n=90) were obtained during surgery from breast cancer patients (Lublin, Poland). The average follow-up period was 70 months. After pathologic review, a tissue microarray (TMA) was constructed from the most representative area of paraffin-embedded breast cancer tissues. The TMAs were used for specific immunohistochemistry staining. Clinic and pathological information is listed in Table 4.

### Immunohistochemistry and Immunofluorescence Staining

Tumor tissues were stained for carbonic anhydrase IX (CAIX) (#5649, Cell Signaling Technology) and SLC6A14 (HPA003193, Sigma) expression. After baking in a thermostat drier at 60° C. for an hour, tissue sections were deparaffinized with rehydrated and xylene. 3% (vol/vol) hydrogen peroxide was used to quench the endogenous peroxidase activity for 10 minutes, followed by four 3-minute washes with double-distilled water. Subsequently, the slides were immersed in AR6 buffer (AR6001KT, PerkinElmer) and heated in a microwave oven for 45 seconds at P10 and 15 minutes at P2. After four 3-minute washes with PBS and pretreatment with PBS containing 5% (wt/vol) bovine serum albumin for 30 minutes, the sections were incubated in a humidified box at 4° C. overnight with the primary antibody. After four 5-minute washes with PBS, the sections were incubated with a biotinylation-treated second antibody (MP-7401, ImmPRESS) for 30 minutes at 37° C., followed by another four 5-minute washes with PBS. Reaction product was visualized using diaminobenzidine (DAKO) for 2 minutes, and counterstained with hematoxylin. Sections were left to air-dry and mounted with permanent mounting medium. Expression of SLC6A14 and CAIX was scored using the H-score method^{7,8}. H-score method took the percentage of positive cells (0-100%) and each staining intensity (0-3+) into account. A final score was calculated on a continuous scale between 0 and 300 using the following formula: H-score=[1×(% cells 1+)+2×(% cells 2+)+3×(% cells 3+)]. Based on the median value of SLC6A14 expression, patients were divided into high and low expression groups.

For immunofluorescence staining, cells were fixed with 4% paraformaldehyde for 10 minutes at room temperature. After washing with PBS 3 times, cells were permeabilized with 0.5% Triton X-100 for 10 minutes at room temperature, and blocked with 3% BSA for 30 minutes at 37° C. The coverslips were incubated with primary antibodies at 4° C. overnight. Following washing with PBS additional 3 times, coverslips were stained with appropriate Alexa fluor conjugated secondary antibodies for 30 minutes at 37° C., then washed three times with PBS and mounted on glass slides. For F-actin staining, the coverslips were incubated with Alexa Fluor™ 647 Phalloidin (A22287, Invitrogen) for 60 minutes at 37° C., then washed three times with PBS and mounted on glass slides. Images were acquired using a confocal laser scanning microscope (Nikon Eclipse Ti Confocal System, Japan). The negative control samples were treated with mouse or rabbit IgG antibodies.

### Mouse Models

Six to eight week-old female BALB/cJ mice were purchased from The Jackson Laboratory. The work was approved by the Institutional Animal Care and Use Committee at the University of Michigan. Slc6a14^{-/-} and Slc6a14^{+/+} 4T1 tumor cells were inoculated into the fourth right mammary fat pad of BALB/cJ mice. Tumor size was measured every three days using calipers fitted with Vernier scale. Tumor volume (TV) was calculated using the formula: TV=0.5×L×W². In some cases, single cell suspensions were made from spleen and tumor tissues for immune phenotype and function analysis.

For CD8⁺ T cell depletion experiments, tumor-bearing BALB/c mice were intraperitoneally (i.p.) injected with 100 μg anti-CD8b (clone 53-5.8; Bio X Cell) on day −1, 3, 7, and 11 post tumor cell inoculation.

For the in vivo tumor hypoxia visualization, 2*10⁴ 4T1 tumor cells were inoculated into the fourth right mammary fat pad of 6-8 week-old female BALB/cJ mice. Three weeks after inoculation and a tumor volume reaching about 500 mm³, the hypoxic cell marker pimonidazole hydrochloride [1-[(2-hydroxy-3-piperidinyl) propyl]-2-nitroimidazole hydrochloride; Hypoxyprobe] was injected (i.p.; 20 mg/mL in physiologic saline; 60 mg/kg; nominal injected volume, 0.1 mL) 10 minutes before animals were euthanized. Tumor tissues were immediately dissected and embedded in paraffin, and cut in 6 μm thick sections. Sections were co-stained with FITC-conjugated anti-pimonidazole monoclonal antibody (green; HP2-100 kit, Hypoxyprobe) and anti-SLC6A14 antibody (HPA003193, Sigma). Donkey anti-Rabbit IgG (H+L) secondary antibody with Alexa Fluor 546 (A10040, Invitrogen) was used at a concentration of 4 μg/mL in phosphate buffered saline containing 0.2% BSA for 45 minutes at room temperature for detection of SLC6A14 in the cytoplasm (red). Cell nuclei were labeled with DAPI (blue; Invitrogen Corp). The confocal laser scanning microscope (Nikon Eclipse Ti Confocal System, Japan) was used to scan tumor tissue sections. At least 6 independent fields were analyzed from each tissue section in individual tumors.

For the therapeutic experiments, 2*10⁴ 4T1 cells were inoculated into the fourth right mammary fat pad of BALB/cJ mice. On day 3, mice were treated (i.p.) with 200 μg of anti-PD-L1 (Bio X Cell) every 3 days, and/or with 250 μg of 4-HAP (i.p., 278564, Sigma) every other day, and/or α-MT (2 mg/mL, M8377, Sigma) in drinking water. Tumor diameters were measured using calipers.

### Cycloheximide (CHX)-Chase Analysis

SLC6A14 protein stability was analyzed by the CHX-chase analysis. 12 hours after exposure to hypoxia, MDA-MB231 cells were further cultured in normoxic and hypoxic condition for 0, 1, 2, 4, and 6 hours in the presence of CHX (100 μM). Cells were harvested at each time point. Total protein (60 μg) was subjected to immunoblotting with anti-SLC6A14, anti-HIF1α, and anti-β-actin antibodies. Expression levels of SLC6A14 were quantified by densitometry. SLC6A14 protein half-lives were calculated from the slope of the semi-logarithmically transformed the best fit line. The decay curves were analyzed individually using linear regression of protein amount, and expressed as a percentage of protein remaining versus time.

### Cell Membrane Pore Propidium Iodide Incorporation

Tumor cell membrane pore propidium iodide (PI) incorporation was evaluated by fluorescence microscopy and flow cytometry. For perforin-mediated membrane pore formation, PY8119 tumor cells were cultured with perform in the presence of propidium iodide (PI) (100 μM)(Life Technologies). PI incorporated cells were analyzed by flow cytometry analysis or were imaged with a fluorescence microscope at a 10× and 30× objective lens (Olympus). The percentage of PI⁺ tumor cells was quantified. For CTL-mediated membrane pore formation, PY8119 tumor cells stably expressing with scrambled sh-RNAs or sh-Slc6a14 were labeled with carboxyfluorescein succinimidyl ester (CFSE, 2 μM) in serum-free DMEM according to the manufacturer instructions (Thermo Fisher, C34554). Then, the cells were pulsed with ovalbumin (OVA) peptide for another hour. OVA+PY8119 cells (10⁴) were co-cultured with purified CD8+OT1 cells (10⁵) for 4 hours. PI was added to the medium. PI+ tumor cells were similarly analyzed by flow cytometry or imaged with a fluorescence microscope.

### Cell Death Measurement

Tumor cells were exposed to hypoxia for 24 hours, or the indicated conditions. Then, tumor cells were co-incubated with perform (APB317Hu01, Cloud-Clone) and granzyme B (2906-SE-010, R&D system) for 10 minutes. PI (10 μg/ml) was added into the culture. PI⁺ cells were measured by flow cytometry.

### AFADESI-MSI Analysis

In vivo tumor cell amino acid-uptake was detected with the airflow-assisted desorption electrospray ionization mass spectrometry imaging system (AFADESI-MSI). Fresh tumor tissues were snap-frozen in liquid nitrogen for 10 seconds after resection and stored at −80° C. Tumor tissues were sliced to 15 μm in thickness using a CM 1860 cryostat microtome (Leica Microsystem, Wetzlar, Germany) and thaw-mounted onto microscope slides (Fisher Superfrost Plus). Before AFADESI-MSI analysis, the frozen tissue sections were first dried in a vacuum container at −20° C. for 1 hour and then transferred to room temperature for another hour. AFADESI-MSI analysis and data processing were carried out as previously described³⁶. Briefly, AFADESI-MSI analysis was performed in both positive and negative ion mode on a Q-Exactive mass spectrometer (Thermo Scientific, Bremen, Germany) over an m/z range of 70-1000 at a nominal mass resolution of 70,000. A mixture of acetonitrile and water (8:2, v/v) was used as the spray solvent with a flow rate set to 5 μL/min. The sprayer and transport tube voltages were set at 7000 V and 0 V in positive ion mode and at −7000 V and 0 V in negative ion mode. The extracting gas flow was 45 L/minute, and the capillary temperature was 350° C. The MSI experiments were performed by continuously scanning the tissue surface in the x-direction at a constant rate of 200 μm/second with a 200 μm vertical step, separating the adjacent lines in the y-direction. The spatial resolution of AFADESI-MSI system was approximately 100 μm in this study. The maximum cross tissue section areas were scanned and analyzed in individual tumors. Tumor amino acid-uptake was quantified via the intensity of individual amino acid signal per pixel.

### RNA Extraction and Real-Time PCR

Total RNA was extracted using TRIzol™ Reagent (ThermoFisher Scientific, USA). RNA was quantified by using NanoDrop spectrophotometer. Reverse transcription of the total cellular RNA was carried out using oligo (dT) primers and M-MLV reverse transcriptase (ThermoFisher Scientific, USA). RT-PCR primers are listed (Table 6). PCR was performed using QuantStudio 3 Real-time PCR system (Appliedbiosystems, Thermo Fisher Scientific, USA).

### Chromatin Immunoprecipitation (ChIP)

ChIP was performed using the SimpleChIP Enzymatic Chromatin IP Kit (Magnetic Beads, Cell Signaling Technology, Danvers, MA) following manufacturer instructions. ChIP primers are listed (Table 6). The resulting precipitated DNA samples were quantified by real-time PCR.

### Optical Tweezers Microscopy (OTM)

OTM was established to measure tumor cell stiffness. A modular OTM (Thorlabs—OKBT) was adapted for axial cell indentation and force measurement. Briefly, a collimated laser beam (YLM-5-IPG Photonics—Yb fiber laser 1064 nm) was focused through a 100× objective (Nikon, N.A.=1.25, oil immersion) to produce a stable optical trap for 3 μm diameter silica microbeads (Bangs Laboratories) dispersed in the medium. During data acquisition, the cells were maintained at 37° C. by an integrated temperature controller unit (RS Components). The sample was imaged through the trapping objective and a tube lens by a CMOS camera (Thorlabs—DCC1240C). A microbead probe was individually trapped and positioned above the cell nucleus area of a selected cell using a piezoelectric NanoMax 3-axis flexure stage (Thorlabs). The position of the microbead in the trap was tracked by back focal plane interferometry method using the laser light scattered by the trapped bead. The trap height was in the range 3-6 μm from the cover-slip. The trap stiffness was calculated using the equipartition theorem and the power spectrum density methods considering the stiffness variations due to spherical aberrations and neighborhood of the cover-slip. Adjusting the laser power, trap stiffness was set to 0.015 pN/nm. To indent the cell, the stage on which the sample was clamped was moved vertically following a sinusoidal wave of 1 μm amplitude and 0.2 Hz frequency, while the laser focus was kept at a fixed position. The cell intercepted and interacted with the microbead in the second half of the sine period, thus causing a vertical displacement of the microbead with a proportional force. Tumor cell stiffness was determined and expressed as megapascal (MPa) per site. At least 15 cells per condition were analyzed.

### Atomic Force Microscopy (AFM)

AFM was used to measure tumor tissue stiffness in vivo. MFP-3D AFM (Asylum Research, Santa Barbara, CA) combined with a Nikon Ti inverted optical microscope (Nikon, Melville, NY) was used to optically align the probe to the cells. The probes used in this study were MCST-AUHW (Bruker, Camarillo, CA) with a nominal spring constant of 0.03 N/m. To simplify the contact geometry and minimize the lateral strain of the sample during indentation, the cantilever tip was modified by attaching a plain silica microsphere of diameter 4.7 mm. Measurements were conducted in cell culture media at room temperature, with cells plated on the glass bottom of the Fluorodish. To eliminate the confounding effects of neighboring cells on cytoskeleton arrangement and morphology, single cells were measured.

Prior to cell measurements, the cantilever was calibrated on the glass bottom of the Fluorodish using the thermal vibration method with the resultant thermal spectrum fitted with Lorentzian function to determine the spring constant. The cells were indented approximately over the perinuclear region of individual cells. The indentation depth was chosen to be at least 1 mm in order to better simulate deformations that occur physiologically. The force versus indentation curves from each measurement were analyzed using a Hertzian contact model to obtain the Young's modulus of each cell.

For in vivo tumor cell stiffness detection, fresh tumor tissues were excised and prepared immediately as frozen slices. AFM was performed within hours following tissue removal. The deflection sensitivity was determined in fluid using glass substrates as an infinitely stiff reference material. AFM at room temperature involved recording up to 6×6 point grids (36 force-displacement curves per map) was recorded. Force-volume maps spaced at 500 μm apart were acquired in a systematic manner across the entire sample surface. Individual force curves consisted of 36 data points with a Z piezo-displacement between 5 and 8 μm, which were collected at 0.8-1 load/unload cycles per second. The maximum applied loading force was set to 1.8 nN; this gave indentation depths that varied between approximately 150 and 3,000 nm, depending on the intrinsic mechanical differences within each biopsy. Force curves were analyzed using the Oliver and Pharr method. Tumor stiffness was determined and expressed as log 10 (pascal) per site. At least 24 sites per tumor tissue were analyzed.

For tumor cellular membrane pore visualization, force-distance curve-based AFM (FD-based AFM) was employed using a Dimension ICON AFM (Bruker, Santa Barbara, USA) set to PeakForce Tapping mode. The AFM was equipped with a 90 μm piezoelectric scanner. The parameters were set as previously described³⁸. AFM images were analyzed and processed with Nanoscope Software (Bruker, Karlsruhe, Germany). Each tapping mode image was flattened to measure the diameter and depth of each pore. A pore was defined as a cavity deeper than 10 nm in the plasma membrane. Pore size was determined by pore diameter and depth. Pore diameter was measured from both the longest and shortest diameters around the pore. Pore depth was measured from the highest protruding rim relative to the lowest concave edge. Pore diameter, depth, and number were quantified in a cellular membrane area of 5×5 μm² in 5 cells.

### Intracellular Glutamine Measurement

Intracellular glutamine was measured in cell lysate using the glutamine and glutamate-Glo Assay (courtesy of Promega). Luminescence was measured using a SpectraMax miniMax 300 imaging cytometer, and data were normalized for cell number.

### Immunoblot

Cell lysates were prepared in RIPA buffer (Thermo Fisher Scientific, USA). The protein concentrations of cell lysates were determined by BCA protein assay kit (Thermo Scientific, USA). Equivalent amounts of total cellular protein were separated by SDS PAGE and transferred to PVDF membranes. The proteins were detected with specific antibodies (Table 7) and developed using ChemiDoc™ Imaging System (Bio-Rad, USA).

### Flow Cytometry Analysis (FACS)

For cell nuclear and chromosome counterstain detection, cells were treated and stained with propidium iodide (PI, Thermo Fisher Scientific) and directly run on Fortessa flow cytometry (BD Biosciences, San Jose). For cell surface MHC-I detection, cells were treated and stained with H2Kb antibody (BD Biosciences). Single cell suspensions were prepared from fresh mouse tumor tissues and lamina propria mononuclear cells (LPMC). Cells were stained with fluorescence conjugated anti-CD3, anti-CD90, and anti-CD8 mAbs. Cytokine expression was determined by intracellular staining with anti-granzyme B, anti-TNFα, and anti-IFNγ mAbs (BD, USA). Based on CFSE-labels and PI incorporation, perform- and CTL-mediated tumor cell membrane pore formation was analyzed by FACS. All samples were read on Fortessa flow cytometry and data were analyzed with DIVA software (BD Biosciences). Antibodies used for flow cytometry analysis are listed in Table 7.

### RNA-Sequence and Proteomic Studies on Human Tumors

For evaluating the role of hypoxia in stiffness gene signature in human breast cancer cells, MCF-7 cells were exposed to hypoxia or normoxia for 48 hours. The Gene Set Enrichment Analysis (GSEA) was applied to the RNA-seq data from these tumor cells (GSE47533). Gene set was considered significant when the false discovery rate (FDR) was less than 0.25.

For evaluating the role of SLC6A14 in stiffness gene signature in breast cancer patients, The Cancer Genome Atlas (TCGA) gene dataset with 1218 breast cancer samples was downloaded from the UCSC Xena. Of these cases, the top 20% high and 20% low SLC6A14 transcripts were denoted as “SLC6A14 high” and “SLC6A14 low” groups. Using the signal-to-noise measure in the GSEA, 20,501 genes were ranked by their association with the breast cancer groups (SLC6A14 low vs. SLC6A14 high). Pre-ranked GSEA was performed using a dataset of the 79 stiffness-associated genes (www.cbioportal.org/) (Table 5).

For determining the impact of stiffness genes on checkpoint therapy efficacy, the tumor proteomic analysis was performed in 67 melanoma patients having received anti-PD1 therapy⁴⁷. Expression of tumor MYH9 and MYLK protein was determined in post-therapy melanoma biopsies. Patients were divided into 2 groups based on the median levels of MYH9 and MYLK. The rates of clinical responses, including complete and partial responses (CR and PR) and progressive diseases (PD), were compared in these 2 patient groups.

OT-I Cell Isolation and Co-Culture with OVA+ Tumor Cells

OT-I transgenic mice, C57BL/6-Tg (TcraTcrb) 1100Mjb/J were purchased from The Jackson Laboratory. Spleen was homogenized. Single cells were suspended in 2 ml Red Blood Cell Lysis Buffer (Sigma-Aldrich) for 1 minute. Splenocytes were pelleted, washed, and resuspended at 2×10⁶ cells per ml in RPMI culture medium containing 1 μg/ml OVA257-264 peptide, 5 μg/ml mouse recombinant IL-2, and 40 μM 2-mercaptoethanol. The cells were incubated at 37° C. for 5 days.

For the co-culture of OT-I cells and OVA+ tumor cells, splenocytes from OT-I transgenic mice were activated with OVA for 5 days. OT-I cells were purified using EasySep mouse CD8+ T Cell Isolation Kit (Stemcell). OVA+PY8119 tumor cells were seeded overnight; OT-I cells were then added into the culture for 24 hours or the indicated time points. All cells were collected by trypsinization and analyzed by flow cytometry or fluorescence microscope.

### Quantification and Statistical Analysis

Comparisons between two groups were performed by using unpaired Student's t-test or one-way ANOVA. Correlations between groups were determined by Pearson's correlation test. Tumor growth in different groups was tested by ANOVA models. Pearson correlation was used to evaluate the association between two genes or two proteins. Survival rate was estimated by the Kaplan-Meier method and compared using the log-rank test. P<0.05 was considered statistically significant. Analyses were performed using GraphPad Prism 8.0 (GraphPad Software, Inc., La Jolla, USA) software.

### Results

### Hypoxia Endows Tumor Resistance to CTL-Killing Via SLC6A14-Mediated Amino Acid Uptake

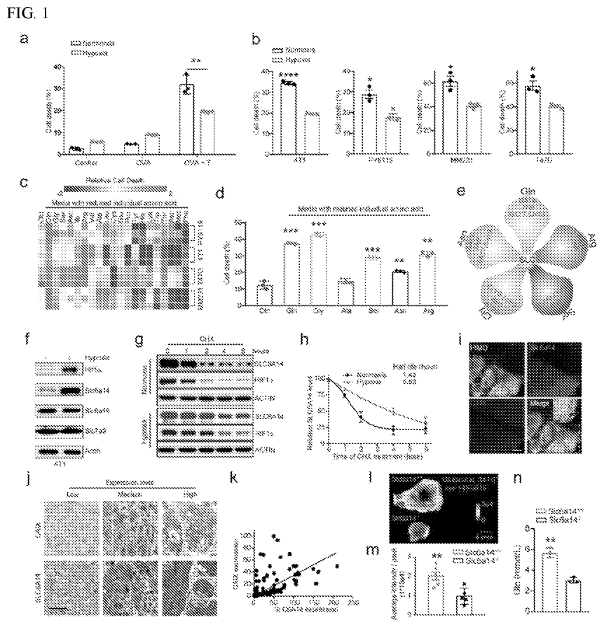

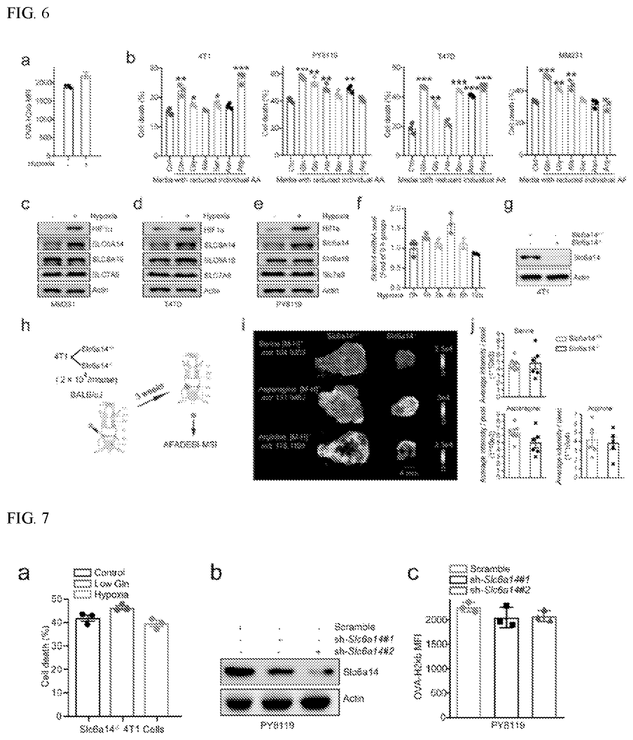

To explore a potential role of hypoxia in antigen-specific CTL-mediated tumor killing, ovalbumin (OVA) expressing PY8119 mouse breast cancer cells were exposed to hypoxia and then cultured with activated OVA-specific CTLs, OT-I cells. Hypoxic exposure resulted in a decrease in OT-I-mediated tumor killing (FIG. 1a). Comparable levels of MHC-I-OVA peptide complexes were detected on the membrane surface of tumor cells exposed to hypoxia and normoxia (FIG. 6a), indicating that hypoxia did not induce a loss of antigen presentation. CTL-derived perform mediates pore formation on target cell membrane, which facilitates granzyme B enter into target cells and initiates the killing event³⁰. To test if hypoxia altered perforin-mediated tumor cell membrane pore formation, four breast cancer cells (including mouse PY8119 and 4T1 cells, and human MDA-MB231 and T47D cells) were exposed to hypoxia, cultured with perform and granzyme B, and the tumor cell death was assayed in a CTL-free system (FIG. 1b). A decrease in perform and granzyme B-induced cell apoptosis was observed in both mouse and human breast cancer cells previously exposed to hypoxia, as compared to normoxia exposed cells (FIG. 1b). Thus, hypoxia may endow cancer cell resistance to perforin-mediated pore formation and CTL-mediated killing.

The underlying mechanisms whereby hypoxia endows tumor cell resistance to CTL-mediated killing was investigated. As a survival adaptive mechanism, tumor cells alter particular amino acid uptake and utilization in response to hypoxic stress^{2,31}. Specific amino acid metabolism affects T cell-mediated anti-tumor immunity^{27,32-34}. It was hypothesized that particular amino acids may be involved in hypoxia-directed tumor cell receptivity to CTL-mediated killing. To test this hypothesis, the aforementioned 4 breast cancer cell lines were cultured with reduced concentrations of individual amino acids (amino acid starvation), including all 20 individual amino acids, and added perforin and granzyme B in the culture to induce tumor cell death. Flow cytometry demonstrated that breast cancer cells responded differentially to individual amino acid starvation, as shown by propidium iodide (PI)⁺ tumor cells (FIG. 1c and FIG. 6b). The starvation of 6 individual amino acids (including alanine, arginine, asparagine, glutamine, glycine, and serine) caused significant apoptosis in more than 2 breast cancer cell lines among all 4 tested tumor cell lines (FIG. 1c and FIG. 6b). Then, OVA expressing PY8119 breast cancer cells were incubated with reduced concentrations of each of the 6 individual amino acids and subsequently cultured with OT-I cells. It was found that starvation of all but alanine enhanced CTL-mediated tumor killing capacity, as shown by PI⁺ tumor cells (FIG. 1d). Additionally, glutamine starvation resulted in the most pronounced tumor cell death in all 4 tumor cell lines (FIG. 1c and FIG. 6b). Thus, the data indicates that hypoxia may alter amino acid (particularly glutamine) uptake in tumor cells, inducing their resistance to CTL-killing.

Amino acid uptake is often mediated by specific transporters—solute carriers (SLCs). Thirty-one SLC members are potentially involved in the uptake of the aforementioned 5 individual amino acids, including glutamine, glycine, serine, asparagine, and arginine³⁵. Four members (including SLC6A14, SLC6A17, SLC6A19, and SLC7A9) are able to uptake more than 3 to 5 amino acids35 (FIG. 1e). SLC6A14, SLC6A19, and SLC7A9 were studied; SLC6A17 was excluded due to its exclusive expression in the brain³⁶. As both hypoxia (FIG. 1a-b) and specific amino acid (e.g., glutamine) starvation (FIG. 1c-d and FIG. 6b) affected tumor cell receptivity to CTL-killing, it was contemplated that hypoxia altered amino acid transporter SLC expression to regulate tumor cell receptivity to CTL-killing. In support of this, it was uncovered that hypoxia strongly induced expression of Slc6a14, but not Slc7a9 and Slc6a19, in 4T1 tumor cells (FIG. 1f). Similar results were obtained in MDA-MB231 (FIG. 6c), T47D (FIG. 6d), and PY8119 (FIG. 6e) cells. Hypoxia had minimal effect on Slc6a14 transcripts in 4T1 cells at different time points (FIG. 6f). The cycloheximide (CHX) chase assay revealed that hypoxia prolonged the half-life of SLC6A14 protein from approximately 1.5 to 5.5 hours (FIG. 1g-h). Thus, hypoxia strongly induces and maintains SLC6A14 expression in breast cancer cells.

To validate this observation in vivo, 4T1 tumors were established in BALB/cJ mice. Pimonidazole is an effective and nontoxic exogenous 2-nitroimidazole hypoxia marker. 4T1 tumor bearing mice were treated with pimonidazole, then 4T1 tumor tissues were stained with anti-pimonidazole and anti-SLC6A14 monoclonal antibodies (mAbs). Highly consistent positive staining areas were detected between SLC6A14 and pimonidazole in serial tissue sections (FIG. 1i). Furthermore, immunohistochemistry staining on human breast cancer tissues with anti-SLC6A14 and anti-CAIX (Carbonic anhydrase IX, a hypoxic marker) identified a positive correlation between CAIX and SLC6A14 expression in human breast cancer tissues (FIG. 1j-k). Thus, hypoxia induces tumor SLC6A14 protein in murine breast cancers and in patients with breast cancer.

To examine a role of SLC6A14 in tumor amino acid uptake in vivo, Slc6a14 knockout 4T1 tumor cell clones (referred to as Slc6a14^{-/-}) using Cas9 knock out technology were established (FIG. 6g), and inoculated Slc6a14^{-/-} and Slc6a14^{+/+} 4T1 tumor cells into syngeneic BALB/cJ mice. An airflow-assisted desorption electrospray ionization mass spectrometry imaging system (AFADESI-MSI)³⁷ was used to evaluate the effect of Slc6a14 on tumor glutamine, serine, asparagine, and arginine uptake in vivo (FIG. 6h). AFADESI-MSI demonstrated an obvious decrease in glutamine signals per pixel (FIGS. 1l-m) (but not serine, asparagine, and arginine) in Slc6a14^{-/-} 4T1 tumor cells compared to Slc6a14^{+/+} 4T1 tumor cells in vivo (FIG. 6i-j). A reduction of glutamine contents in Slc6a14^{-/-} 4T1 cells as compared to Slc6a14^{+/+} 4T1 cells in vitro was confirmed (FIG. 1n). Thus, hypoxia induces tumor SLC6A14 expression, enhances amino acid (particularly glutamine) uptake, and abolishes tumor receptivity to CTL-killing.

### SLC6A14 Diminishes Tumor Receptivity to CTL-Killing Via Altering Membrane Pores

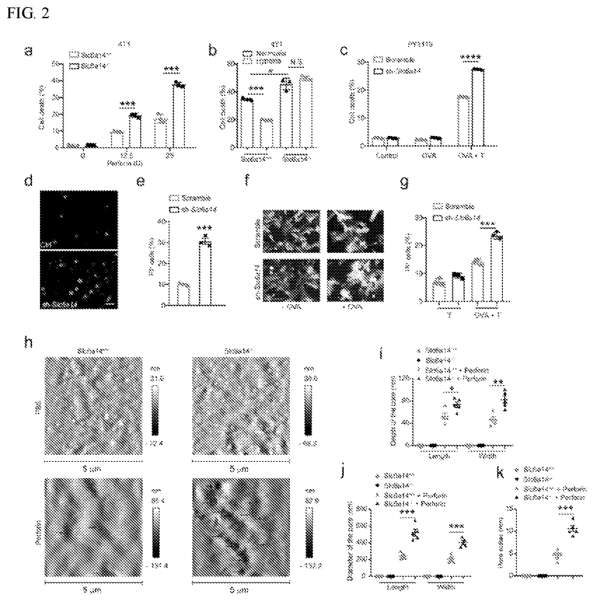

Hypoxia induced tumor SLC6A14 expression and promoted tumor resistance to CTL-mediated killing (FIG. 1). It was determined if hypoxia contributed to tumor resistance to CTL-killing via SLC6A14 by culturing Slc6a14^{-/-} and Slc6a14^{+/+} 4T1 cells with perform and granzyme B. Perforin and granzyme B induced 4T1 cell death in a perform dose-dependent manner (FIG. 2a). Higher levels of cell death were detected in Slc6a14^{-/-} 4T1 cells than Slc6a14^{+/+} 4T1 cells (FIG. 2a). Then, Slc6a14^{-/-} and Slc6a14^{+/+} 4T1 cells were exposed to hypoxia. Hypoxia resulted in reduced Slc6a14^{+/+} 4T1 cell death, but had no effect on Slc6a14^{-/-} 4T1 cells (FIG. 2b). To further connect the involvement of glutamine in this setting, perforin and granzyme B-induced cell death was examined in Slc6a14−/− 4T1 cells in normoxia and hypoxia with or without glutamine starvation. Glutamine starvation or hypoxia failed to additionally affect Slc6a14^{-/-} 4T1 cell death (FIG. 7a). The study was extended to a CTL-killing assay using an additional breast cancer cell line—PY8119 cells. Slc6a14 was knocked down with short hairpin RNA (sh-Slc6a14) in OVA⁺-PY8119 target cells (FIG. 7b), then cultured with OT-I cells. An increase in OT-I-mediated tumor killing in sh-Slc6a14 tumor cells as compared to scrambled controls was observed (FIG. 2c). Scramble tumor cells and sh-Slc6a14 tumor cells expressed similar levels of MHC-I-OVA peptide complexes on their membrane surfaces (FIG. 7c). Thus, hypoxia contributes to tumor resistance to CTL-killing via SLC6A14-mediated glutamine uptake.

CTL-derived perform initiates killing by inducing pore formation on the target cell membrane. The effect of SLC6A14 on perforin-mediated pore formation on tumor cells was investigated by exposing control or sh-Slc6a14 PY8119 cells to perform in the presence of saturated PI. PI penetrates tumor cells through perforin-induced membrane pores²⁹. Fluorescence microscopic analysis revealed more PI+ cells in sh-Slc6a14 PY8119 cells than scramble controls (FIG. 2d-e). The data support that an increase of number and/or size of perforin-induced membrane pores in in sh-Slc6a14 PY8119 cells. It was next examined if SLC6A14 has an impact on CTL-mediated membrane pore formation. OVA-expressing sh-Slc6a14 PY8119 cells were labeled with carboxyfluorescein succinimidyl ester (CFSE), cultured with OT-I cells in the presence of PI, and PI incorporation in OVA-expressing CFSE-labelled PY8119 cells was assayed. Higher percentages of PI+ cells were found in OVA-expressing CFSE-labelled sh-Slc6a14 PY8119 cells than in that of control cells (FIG. 2f-g). Furthermore, atomic force microscopy (AFM) was used to visualize tumor cell membrane pores³⁸, and directly compare perforin-drilled membrane pores in Slc6a14^{+/+} and Slc6a14^{-/-} 4T1 cells. AFM images revealed more and larger pores in Slc6a14^{-/-} 4T1 cells than Slc6a14^{+/+} 4T1 cells, drilled by perforin (FIG. 2h-k). Thus, SLC6A14 abolishes tumor receptivity to CTL-killing via altering membrane pore formation.

### Hypoxia Reduces Cancer Stiffness by Targeting Myosin II Via SLC6A14

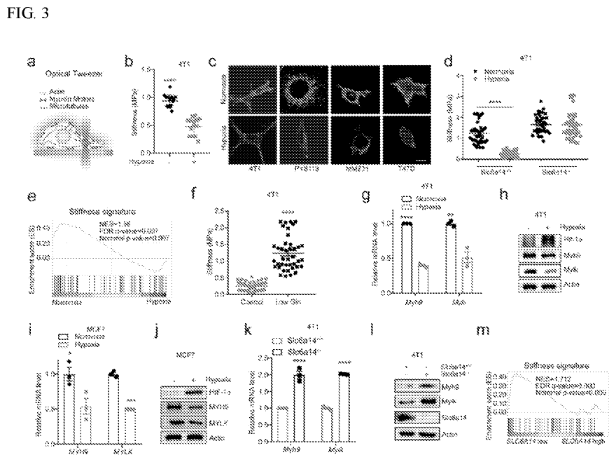

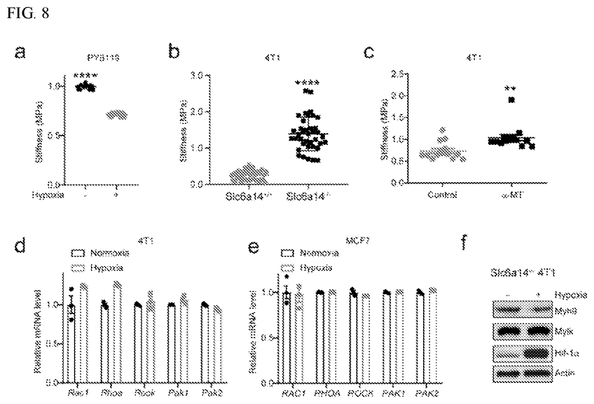

Target cell membrane mechanical tension plays a critical in efficient pore formation mediated by CTL-derived perforin²⁹. It was investigated whether hypoxia altered tumor cell membrane mechanical tension to reduce perforin-drilled membrane pores, thereby causing an impaired tumor receptivity to CTL-killing. To examine this query, an Optical Tweezers Microscopy (OTM) technique was developed to measure cellular membrane mechanical stiffness (FIG. 3a)³⁹. It was found that hypoxia induced a decrease in cellular membrane stiffness in 4T1 and PY8119 cells as compared to normoxia (FIG. 3b and FIG. 8a). Cortical filamentous-actin (F-actin) distribution positively correlated with cellular stiffness⁴⁰. Hypoxia caused a loss of the cortical structure of F-actin in several mouse (4T1 and PY8119) and human (MDA-MB231 and T47D) breast cancer cell lines as compared to normoxia (FIG. 3c). The data indicates that hypoxia alters the mechanical properties of cancer cells.

Given that hypoxia induced tumor SLC6A14 expression, it was contemplated that SLC6A14 was involved in controlling cancer cell stiffness. In support of this, an increase in cellular stiffness was detected in Slc6a14^{-/-} 4T1 cells compared to Slc6a14^{+/+} 4T1 cells in normoxia (FIG. 8b). Furthermore, functional inhibition of SLC6A14 with α-methyl-DL-tryptophan (α-MT), a biochemical blocker for SLC6A1441, enhanced 4T1 cell stiffness (FIG. 8c). Hypoxia reduced cellular stiffness in Slc6a14^{+/+} 4T1 cells, but not in Slc6a14^{-/-} 4T1 cells (FIG. 3d). Additionally, gene set enrichment analysis (GSEA) revealed that cell stiffness gene signature was enriched in MCF7 cells cultured under normoxic condition as compared to hypoxic condition (FIG. 3e). Thus, hypoxia alters cancer cell stiffness via SLC6A14. As glutamine uptake via hypoxia-induced SLC6A14 affected tumor cell receptivity to CTL-killing, the involvement of glutamine in cancer cell stiffness was examined in the context of SLC6A14. Similar to SLC6A14 deficiency, glutamine starvation increased cell stiffness in Slc6a14^{+/+} 4T1 cells (FIG. 3f). Thus, hypoxia enhances cancer cell stiffness by SLC6A14 via glutamine uptake.

It was next investigated how hypoxia affected tumor cell stiffness via SLC6A14. Myosin II plays a critical role in maintaining the mechanical integrity of cells⁴². Among myosin II heavy chain isoforms, it was observed that hypoxia decreased expression of myosin heavy chain 9 (Myh9) and myosin light chain kinase (Mylk) transcripts (FIG. 3g) and proteins (FIG. 3h) in 4T1 cells. Hypoxia had no effect on expression of other myosin II related genes—including Rac1, Rhoa, Rock, Pak1, and Pak2 in 4T1 cells (FIG. 8d). Similar results were obtained in MCF7 cells (FIG. 3i-j and FIG. 8e). Given that hypoxia reduced tumor cell stiffness via SLC6A14, it was investigated if SLC6A14 regulated MYH9 and MYLK expression. Higher levels of Myh9 and Mylk transcripts (FIG. 3k) and proteins (FIG. 3l) were detected in Slc6a14^{-/-} 4T1 cells than Slc6a14^{+/+} 4T1 cells in normoxic condition. Differing from Slc6a14^{+/+} 4T1 cells (FIG. 3h), hypoxia failed to alter Myh9 and Mylk expression in Slc6a14^{-/-} 4T1 cells (FIG. 8f). Thus, hypoxia reduces breast cancer cell stiffness gene expression via SLC6A14. The relationship between SLC6A14 expression and stiffness genes in patients with breast cancer was next investigated. Based on TCGA data sets in human breast cancer, GSEA analysis revealed that cell stiffness gene signature was enriched in breast cancer patients with low SLC6A14 expression (FIG. 3m). Thus, hypoxia reduces cancer stiffness by targeting MYH9 and MYLK via SLC6A14.

### SLC6A14 Alters FOXO1 Expression to Control Tumor Stiffness Genes

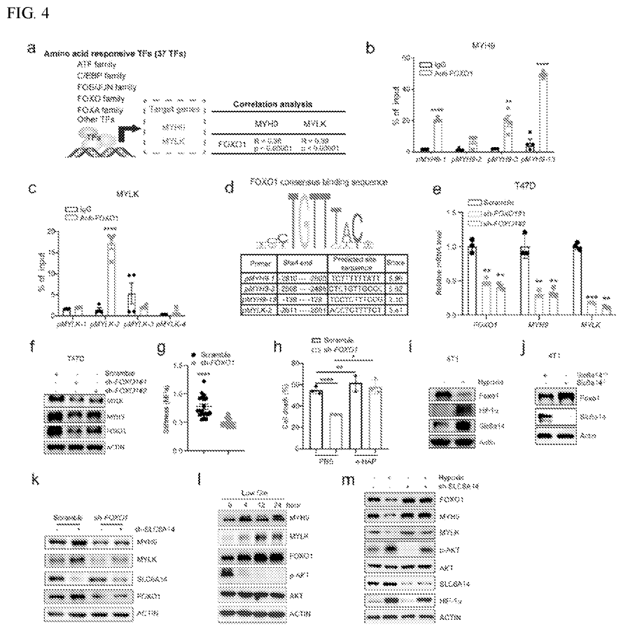

It was explored how hypoxia-induced SLC6A14 regulated MYH9 and MYLK expression. Hypoxia stimulated SLC6A14 expression and altered tumor cell amino acid uptake (FIG. 1). It was contemplated that certain amino acid (e.g. glutamine) responsive transcription factor(s) might contribute to hypoxia-mediated MYH9 and MYLK inhibition. Several transcription factor families can be responsive to amino acids⁴³. Through the large-scale Gene Expression Profiling and Interactive Analysis (GEPIA)⁴⁴, the correlation of 37 amino acid responsive transcription factors with expression of MYH9 and MYLK was analyzed. Among the transcription factors (Table 1), FOXO1 exhibited the highest correlation coefficient values and the lowest P values with both MYH9 and MYLK (FIG. 4a). Thus, 4 sets of experiments to examine the regulatory effect of FOXO1 on tumor stiffness genes in breast cancer cells were performed.

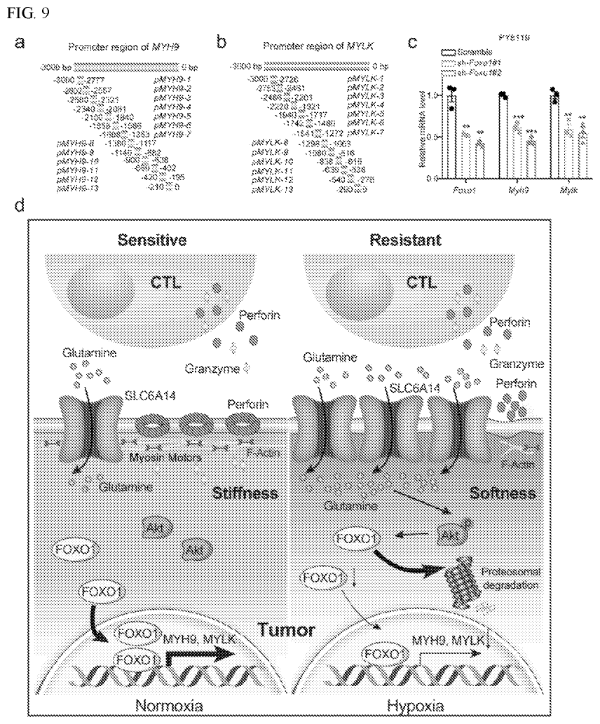

First, it was tested if FOXO1 directly targeted the promoters of MYH9 and MYLK to control their transcription. Multiple primer pairs were used to map out the potential FOXO1 binding regions on the promoter areas of MYH9 and MYLK (FIG. 9a-b). Chromatin immunoprecipitation (ChIP) assays revealed that FOXO1 bound to MYH9 and MYLK promoters in MDA-MB231 cells (FIG. 4b-c and Table 2). In line with this, JASPAR45 database analysis identified the consensus binding motifs in the potential binding regions of MYH9 and MYLK promoter areas (FIG. 4d). Then, FOXO1 was knocked down with shRNAs against FOXO1 in human T47D breast cancer cells or in mouse PY8119 breast cancer cells. Knocking down of FOXO1 resulted in a decrease in both transcript (FIG. 4e and FIG. 9c) and protein (FIG. 4f) levels of MYH9 and MYLK, accompanied by reduced cancer cell stiffness (FIG. 4g). Moreover, FOXO1 knocking down caused an inhibition of perform and granzyme B-induced T47D cell death, which could be rescued by 4-Hydroxyacetophenone (4-HAP) (FIG. 4h). 4-HAP can stiffen tumor cells via enhancing the cortical localization of the mechanoenzyme myosin 1146. Thus, FOXO1 regulates tumor stiffness, and in turn, tumor receptivity to CTL-derived perform membrane drilling.

Next, the relationship between SLC6A14, FOXO1, and stiffness genes in the context of hypoxia was investigated. Wild type (Slc6a14^{+/+}) 4T1 cells were exposed to hypoxia. It was found that along with enhanced Slc6a14 expression, hypoxia resulted in reduced Foxo1 expression in wild type 4T1 cells (FIG. 4i). When compared to Slc6a14^{+/+} 4T1 cells, Slc6a14^{-/-} 4T1 cells expressed higher levels of Foxo1 (FIG. 4j). The data supports that hypoxia reduces FOXO1 expression via SLC6A14. In line with this, knocking down SLC6A14 enhanced FOXO1, MYH9, and MYLK expression in T47D cells. This effect was abrogated in sh-FOXO1 tumor cells (FIG. 4k). Altogether, these results indicate that hypoxia represses FOXO1 via SLC6A14 and FOXO1 transcriptionally controls MYH9 and MYLK expression in breast cancer cells.

Then, a role of glutamine in FOXO1 and stiffness gene expression was investigated. Given that SLC6A14 transported glutamine into tumor cells and affected tumor cell receptivity to CTL-killing, it was hypothesized that FOXO1 was sensitive to glutamine levels and subsequently directed stiffness gene regulation. MDA-MB231 cells were cultured with low levels of glutamine. Tumor cells expressed high levels of FOXO1, MYH9, and MYLK in response to glutamine starvation (FIG. 4l).

Lastly, it wasexplored how glutamine affects FOXO1 expression and stiffness gene expression. It has been reported that amino acids stimulate Akt phosphorylation and activation, thereby promoting FOXO1 degradation^{47,48}. In support of this, it was found that glutamine starvation markedly decreased Akt phosphorylation (FIG. 4l). Furthermore, hypoxia induced Akt phosphorylation, but reduced expression of FOXO1, MYH9, and MYLK. These effects were abrogated in SLC6A14 knocking down MDA-MB231 cells (FIG. 4m). Collectively, the data indicate that hypoxia stimulates SLC6A14, thereby increasing glutamine uptake and activating Akt—in turn reducing FOXO1 expression, and consequently diminishing cancer cell stiffness gene expression. As a result of this molecular cascade reaction, tumor cells become less receptive to CTL-killing due to poor membrane mechanical tension and pore formation (FIG. 9d).

### Targeting Tumor SLC6A14 and Stiffness Affects Tumor Immunity and Immunotherapy

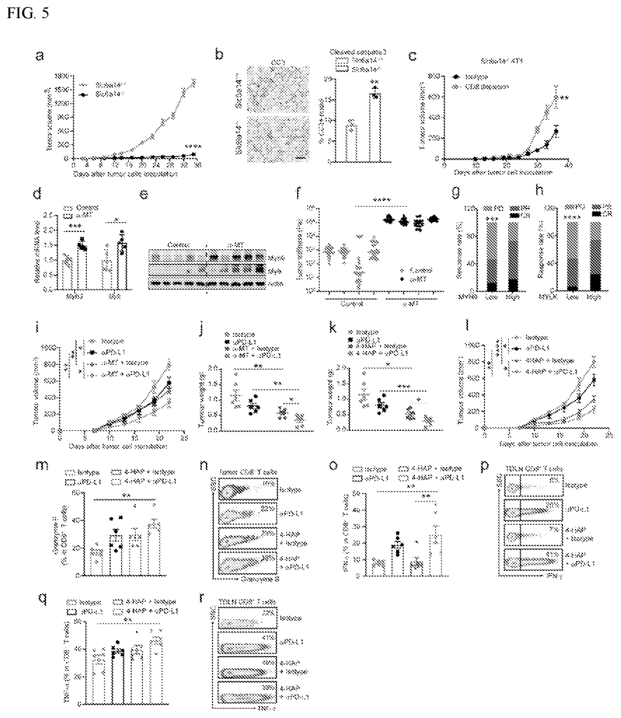

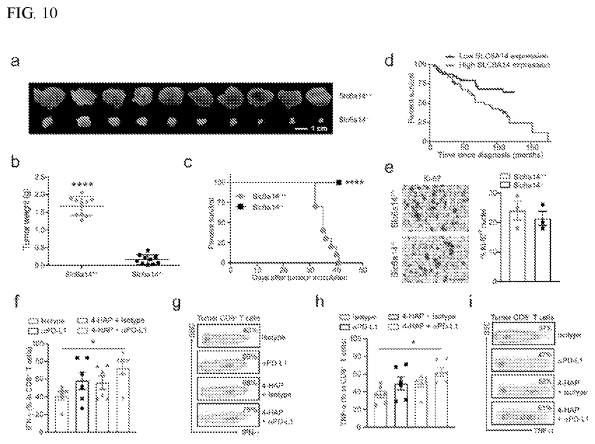

Hypoxia induced tumor SLC6A14 expression and affected tumor cell receptivity to CTL-killing (FIGS. 1 and 2). To explore a potential role of tumor SLC6A14 and stiffness in tumor immunity and immunotherapy in vivo, SLC6A14 and stiffness were genetically and pharmacologically targeted. In the first set of experiments, Slc6a14^{+/+} and Slc6a14^{-/-} 4T1 tumor cells were inoculated into syngeneic BALB/cJ mice. Genetic loss of tumor Slc6a14 caused >90% tumor inhibition in BALB/cJ mice as shown by tumor growth curves (FIG. 5a) and tumor weights (FIG. 10a-b). Mice bearing Slc6a14^{-/-} 4T1 cells experienced longer survival than mice bearing Slc6a14^{+/+} 4T1 cells (FIG. 10c). In line with these data in breast cancer bearing mice, SLC6A14 expression was quantitated in human breast cancer (FIG. 1j) and patients were divided into high and low SLC6A14 expression groups. Kaplan-Meier analysis showed that high expression of SLC6A14 was negatively associated with patient survival (FIG. 10d).

It was next examined whether CD8⁺ T cells actually contributed to diminished tumor growth in mice bearing Slc6a14^{-/-} 4T1 tumor. Ki-67 immunohistochemistry staining was conducted in Slc6a14^{+/+} and Slc6a14^{-/-} 4T1 cancer tissues. Comparable Ki-67+ tumor cells were observed in Slc6a14^{+/+} and Slc6a14^{-/-} 4T1 tumor tissues (FIG. 10e). In contrast, higher levels of cleaved caspase-3 were detected in (CC3⁺) tumor cells in mice bearing Slc6a14^{-/-} 4T1 tumors than Slc6a14^{+/+} 4T1 tumors (FIG. 5b). In line with in vitro CTL-killing data (FIGS. 1 and 2), this in vivo result supports that Slc6a14 expressing cancer cells are resistant to CD8⁺ T cell-induced apoptosis (CTL-killing). To test this, mice bearing Slc6a14^{-/-} 4T1 tumors were treated with anti-CD8 mAb to deplete CD8⁺ T cells. It was found that CD8+ T cell depletion resulted in a dramatic increase in Slc6a14−/− 4T1 tumor growth (FIG. 5c). The data indicated that Slc6a14−/− 4T1 tumor cells are highly receptive to CTL-killing in vivo.

α-MT functionally inhibits SLC6A1441 and enhanced 4T1 cell stiffness in vitro (FIG. 8c). To test if α-MT alters tumor cell stiffness in vivo and affects T cell-mediated tumor immunity, 4T1 tumor bearing mice were treated with α-MT. Treatment with α-MT resulted in reduced levels of Myh9 and Mylk transcripts and proteins in 4T1 tumors in vivo (FIG. 5d-e). Furthermore, α-MT administration increased 4T1 tumor cell stiffness in vivo, as measured by atomic force microscopy (FIG. 5f). To explore the clinical relevance of MYH9 and MYLK expression in immunotherapy, the relationship between MYH9 or MYLK protein expression and checkpoint therapy efficacy in patients with melanoma was examined⁴⁹. The proteomic analysis demonstrated that clinical response rates, including complete and partial responses (CR and PR), were higher in patients with high levels of MYH9 and MYLK protein expression compared to those with low levels of these two proteins (FIG. 5g-h). These results led to a test of the role of α-MT in anti-tumor immunity and immunotherapy. 4T1 tumor bearing mice with were treated with α-MT, anti-PD-L1, and their combination. Single agent therapy (including α-MT and anti-PD-L1) partially and similarly slowed down 4T1 tumor progression, while the combination superiorly inhibited tumor growth (FIG. 5i-j). These results indicate that pharmacologically targeting SLC6A14 synergizes with checkpoint blockade via enhancing tumor stiffness.

In addition to α-MT, 4-HAP can stiffen tumor cells⁴⁶ and promote tumor receptivity to CTL-killing in vitro (FIG. 9g). In exploring whether pharmacologically enhancing tumor cell stiffness improves the efficacy of checkpoint blockade in vivo, 4T1 tumor bearing mice were treated with 4-HAP, anti-PD-L1, and their combination. Single agent therapy with 4-HAP or anti-PD-L1 partially slowed down tumor progression. Their combinatorial treatment was superior in controlling tumor growth as shown by tumor weights and volume (FIG. 5k-l). Compared to single agent therapy, combination therapy resulted in an increase in granzyme B+CD8+ T cells (FIG. 5m-n) and TNF-α⁺ and IFNγ⁺ CD8⁺ T cells (FIG. 10f-i) in both the tumor microenvironment and tumor-draining lymph nodes (FIG. 5o-r). These results indicate that pharmacologically correcting tumor stiffness can synergize checkpoint blockade and enhance tumor immunity. Altogether, the results support that targeting tumor SLC6A14 and stiffness affects immunity and immunotherapy.

TABLE 1
Correlation of amino acid responsive transcription factors with MYH9 and MYLK

| | MYH9 | | MYLK | |
|---|---|---|---|---|
| Transcription factors | R | P | R | P |
| Activating transcription factor 2, ATF2 | 0.23 | 1.2e−14 | 0.16 | 8.8e−08 |
| Activating transcription factor 3, ATP3 | 0.074 | 0.01 [symbol] | 0.14 | 4.2e−0 [symbol] |
| Activating transcription factor 4, ATF4 | 0.3 | 0 | 0.18 | 1.1e−07 |
| Activating transcription factor 5, ATF5 | 0.064 | 0.079 | −0.062 | 0.089 |
| Activating transcription factor 6, ATF6 | 0.19 | 1.6e−10 | 0.075 | 0.013 |
| CCAAT-enhancer-binding protein α, C/EBPα | 0.08 | 0.0084 | −0.012 | 0.69 |
| CCAAT-enhancer-binding protein β, C/EBPβ | 0.051 | 0.095 | 0.083 | 0.0059 |
| CCAAT-enhancer-binding protein δ, C/EBPδ | −0.011 | 0.72 | 0.063 | 0.082 |
| CEBP homologous protein, CHOP | −0.1 | 0.00064 | −0.064 | 0.03 [symbol] |
| cAMP-response element binding protein, CREB | 0.33 | 0 | 0.18 | 2.2e−09 |
| Hepatocyte nuclear factor 1, HNF1 | −0.017 | 0. [symbol] | −0.03 | 0.32 |
| Hepatocyte nuclear factor 4, HNF4 | 0.02 | 0. [symbol] 1 | −0.016 | 0.61 |
| c-Jun | 0.18 | 4.2e−09 | 0.1 [symbol] | 1.1e−07 |
| c-Fos | 0.052 | 0.088 | 0.11 | 0.00015 |
| Jun-B | 0.08 | 0.0086 | 0.0 [symbol] 5 | 0.0053 |
| c-Myc | 0.12 | 3.5e−05 | 0.12 | 9.2e−05 |
| p53 | 0.16 | 7.8e−08 | 0.12 | 5.6e−05 |
| Nuclear factor-κ8, NF-κB | 0.28 | 0 | 0.16 | 1.6e−07 |
| specificity protein 1, SP1 | 0.24 | 4.4e−16 | 0.12 | 4.2e−05 |
| Inhibitor of DNA Binding 1, ID1 | 0.1 | [symbol] e−04 | 0.14 | 4.7e−06 |
| Inhibitor of DNA Binding 2, ID2 | 0.085 | 0.0052 | 0.047 | 0.12 |
| Forkhead box protein O1, FOXO1 | 0.3 [symbol] | 0 | 0.39 | 0 |
| Forkhead box protein O3, FOXO3 | 0.22 | 3.7e−12 | 0.1 [symbol] | 8.7e−07 |
| Forkhead box protein O4, FOXO4 | 0.22 | 3.1e−13 | 0.25 | 0 |
| Forkhead box protein A1, FOXA1 | −0.086 | 0.0046 | −0 [symbol] 18 | 1.5e−09 |
| Forkhead box protein A2, FOXA2 | 0.023 | 0.45 | 0.01 | 0.74 |
| Forkhead box protein A3, FOXA3 | −0.0092 | 0.76 | 0.00016 | 1 |
| Upstream Transcription Factor 1, USF1 | 0.0061 | 0.84 | −0.08 [symbol] | 0.0039 |
| Upstream Transcription Factor 2, USF2 | 0.12 | 1e−04 | −0.018 | 0.56 |
| Nuclear factor erythroid 2-related factor 2, NRF2 | 0.2 | 4.3e−11 | 0.24 | 2.4e−15 |
| Signal transducer and activator of transcription 1, STAT1 | 0.041 | 0.16 | −0.023 | 0.45 |
| Signal transducer and activator of transcription 2, STAT2 | 0.27 | 0 | 0.18 | 2.5e−07 |
| Signal transducer and activator of transcription 3, STAT3 | 0.33 | 0 | 0.2 | 2.2e−11 |
| Signal transducer and activator of transcription 5A, STAT5A | 0.18 | 2.1e−09 | 0.19 | 4.3e−19 |
| Signal transducer and activator of transcription 5B, STAT5B | 0.19 | 3.3e−10 | 0.22 | 4.4e−13 |
| Hypoxia-inducible factor 1-α, HIF1α | 0.27 | 0 | 0.18 | 5.2e−08 |
| Hypoxia-inducible factor 2-α, HIF2α | 0.24 | 4.4e−16 | 0.29 | 0 |

[symbol]

indicates data missing or illegible when filed

TABLE 2
FOXO1 ChIP assay for MYH9 and MYLK

| | | Mean ± s.e.m. | | Fold change | |
|---|---|---|---|---|---|
| Primer pair | ChIP Antibody | (% of Input) | n | of IgG | p |
| pMYH9-1 | IgG | 1.634 ± 0.040 | 4 | | |
| | Anti-FOXO1 | 20.431 ± 0.691 | 4 | 12.506 | <0.0001 |
| pMYH9-2 | IgG | 1.495 ± 0.448 | 4 | | |
| | Anti-FOXO1 | 7.082 ± 1.933 | 4 | 4.738 | |
| pMYH9-3 | IgG | 1. [symbol] 16 ± 0.073 | 4 | | |
| | Anti-FOXO1 | 19.622 ± 3.466 | 4 | 10.2388 | 0.0022 |
| pMYH9-4 | IgG | 0.350 ± 0.231 | 4 | | |
| | Anti-FOXO1 | 3.030 ± 0.6 [symbol] 9 | 4 | 8.660 | |
| pMYH9-5 | IgG | 0.73 [symbol] ± 0.486 | 4 | | |
| | Anti-FOXO1 | 4.795 ± 0.556 | 4 | 6.493 | |
| pMYH9-6 | IgG | 3.400 ± 0.647 | 4 | | |
| | Anti-FOXO1 | 4.991 ± 0.256 | 4 | 1.468 | |
| pMYH9-7 | IgG | 2.138 ± 0.256 | 4 | | |
| | Anti-FOXO1 | 1.9 [symbol] 0 ± 0.326 | 4 | 0. [symbol] 26 | |
| pMYH9-8 | IgG | 6.623 ± 1.648 | 4 | | |
| | Anti-FOXO1 | 6.768 ± 0.230 | 4 | 1.021 | |
| pMYH9-9 | IgG | 3.248 ± 0.692 | 4 | | |
| | Anti-FOXO1 | 4.121 ± 0.150 | 4 | 1.268 | |
| pMYH9-10 | IgG | 2.795 ± 0.708 | 4 | | |
| | Anti-FOXO1 | 4. [symbol] 78 ± 0.278 | 4 | 1.638 | |
| pMYH9-11 | IgG | 1.443 ± 0.216 | 4 | | |
| | Anti-FOXO1 | 1.622 ± 1.27 [symbol] | 4 | 1.124 | |
| pMYH9-12 | IgG | 3.9 [symbol] 5 ± 1.181 | 4 | | |
| | Anti-FOXO1 | 8.163 ± 0.486 | 4 | 2.064 | |
| PMYH9-13 | IgG | 5.962 ± 2.331 | 4 | | |
| | Anti-FOXO1 | 49.296 ± 0.976 | 4 | 8.268 | <0.0001 |
| pMYLK-1 | IgG | 1.634 ± 0.040 | 4 | | |
| | Anti-FOXO1 | 1.193 ± 0.045 | 4 | 1.18 | |
| pMYLK-2 | IgG | 1.495 ± 0.448 | 4 | | |
| | Anti-FOXO1 | 17.271 ± 1.495 | 4 | 11.56 | <0.0001 |
| pMYLK-3 | IgG | 5.318 ± 2.493 | 4 | | |
| | Anti-FOXO1 | 2.150 ± 0.221 | 4 | 0.40 | |
| pMYLK-4 | IgG | 0.328 ± 0.001 | 4 | | |
| | Anti-FOXO1 | 0.814 ± 0.451 | 4 | 2.4 [symbol] | |
| pMYLK-5 | IgG | 0.111 ± 0.000 | 4 | | |
| | Anti-FOXO1 | 0.550 ± 0.032 | 4 | 4.96 | |
| pMYLK-6 | IgG | 13.450 ± 7.407 | 4 | | |
| | Anti-FOXO1 | 15.571 ± 2.934 | 4 | 1.16 | |
| pMYLK-7 | IgG | 0.736 ± 0.584 | 4 | | |
| | Anti-FOXO1 | 0.224 ± 0.020 | 4 | 0.30 | |
| pMYLK-8 | IgG | 0.662 ± 0.1 [symbol] 5 | 4 | | |
| | Anti-FOXO1 | 1.336 ± 0.294 | 4 | 2.02 | |
| pMYLK-9 | IgG | 0.820 ± 0.522 | 4 | | |
| | Anti-FOXO1 | 0.608 ± 0.114 | 4 | 0.74 | |
| pMYLK-10 | IgG | 2.795 ± 0.708 | 4 | | |
| | Anti-FOXO1 | 0.824 ± 0.116 | 4 | 0.29 | |
| pMYLK-11 | IgG | 1.443 ± 0.216 | 4 | | |
| | Anti-FOXO1 | 0.276 ± 0.189 | 4 | 0.19 | |
| pMYLK-12 | IgG | 3.9 [symbol] 5 ± 1.181 | 4 | | |
| | Anti-FOXO1 | 11.288 ± 1.248 | 4 | 2.85 | |
| pMYLK-13 | IgG | 0.69 [symbol] ± 0.233 | 4 | | |
| | Anti-FOXO1 | 0.493 ± 0.010 | 4 | 0.83 | |

* Signal > 5% of input with a fold change of IgG > 5 was considered as positive. [symbol]
indicates data missing or illegible when filed

TABLE 3
Amino acids

| Amino acids | Source | Identifier | Concentration (μg/ml) |
|---|---|---|---|
| Glycine | Sigma | G7126 | 0.2 |
| L-Alanine | Sigma | A7469 | 0.36 |
| L-Arginine | Sigma | A5006 | 4.0 |
| L-Asparagine | Sigma | A0 [symbol] 84 | 1.0 |
| L-Cysteine | Sigma | W326305 | 0.96 |
| L-Cystine | Sigma | 30200 | 1.0 |
| L-Glutamic acid | Sigma | 6106043 | 0.4 |
| L-Glutamine | Sigma | G3126 | 6.0 |
| L-Histidine | Sigma | H8000 | 0.3 |
| L-Isoleucine | Sigma | I2752 | 1 [symbol] 0 |
| L-Leucine | Sigma | L8000 | 1.0 |
| L-Lysine | Sigma | L5501 | 0.64 |
| L-Methionine | Sigma | M9625 | 0.3 |
| L-phenylalanine | Sigma | P2126 | 0.3 |
| L-Proline | Sigma | P8865 | 0.4 |
| L-Serine | Sigma | S4500 | 0.6 |
| L-Threonine | Sigma | T8625 | 0.4 |
| L-Tryptophan | Sigma | T0254 | 0.1 |
| L-Tyrosine | Sigma | T3754 | 0.4 |
| L-Valine | Sigma | V0500 | 0.4 |

[symbol]

indicates data missing or illegible when filed

TABLE 4
Relationship between SLC6A14, CAIX,
and breast cancer patient outcome

| | Overall | | |
|---|---|---|---|
| Parameter | (Percentage) | HR (95% CI) | P |
| Stage | | 1.227~4.582 | 0.01 |
| I-II | 49.4 | | |
| III-IV | 50.6 | | |
| T Stage | | 1.825~6.565 | <0.001 |
| T1-T2 | 75.9 | | |
| T3-T4 | 24.1 | | |
| N Stage | | 1.248~4.761 | 0.009 |
| N0 | 46 | | |
| N1-N3 | 54 | | |
| M Stage | | 0.173~9.367 | 0.813 |
| M0 | 96.6 | | |
| M1 | 3.4 | | |
| Grade | | 1.051~4.683 | 0.037 |
| I-II | 33.3 | | |
| III | [symbol] 6.7 | | |
| Subtype | | 0.621~2.243 | 0.614 |
| Triple negative | 33.3 | | |
| SLC6A14 | | 1.180~4.617 | 0.0105 |
| Low (<median) | 44.8 | | |
| High (>median) | 55.2 | | |

[symbol]

indicates data missing or illegible when filed

TABLE 5
Stiffness gene set

Gene symbol
ABLIM1
ACTR2
ACTR3
ANGPTL2
APOOL
ARHGEF2
CAS
CAU1
CAV1
CD44
CDH1
CDH11
CDH12
CDH16
CDH18
CDH2
CRTC2
CTNNB1
CYP51A1
DHCR24
DHCR7
EBP
FDFT1
FDPS
FLNC
FN1
FSCN2
FSCN3
GGPS1
HMGCR
HMGCS1
HSD1787
ICAM1
IQGAP
KHDRBS2
LIMK1
LOC651621
MMP1
MSMO1
MVD
MVK
MYH2
PAK1
PAK2
PDPN
PPN2
PMVK
PRKAA2
PRMT5
RAC1
RHO
ROCK
ROCK1
RPS4X
SC5DL
SKB1
TM7SF2
TPTE2
TWF1
VEGFA
VIM
WASF1
CTGF
CYR61
AKR1B10
LCP1
FGFR3
ECM1
MAPT
MATN3
CNTNAP2
ERBB4
COL1141
LOXL3
AGTR1
ACP5
LOXL1
CDC42
ROC1

TABLE 6
Sequences for primers and shRNAs

| qPCR primers | Primer sequences |
|---|---|
| Human | |
| SLO6A14-F | TGACCACTTCTGTGCTGGATGG |
| SLO6A14-R | AGCAAGCTCTCCACCATAGCCA |
| MYH8-F | ATCCTGGAGGACCAGAACTGCA |
| MYH9-R | GGCGAGGCTCTTAGATTTCTCC |
| MYLK-F | GAGGTGCTTCAGAATGAGGACG |
| MYLK-R | GCATCAGTGACACCTGGCAACT |
| RAC1-F | CGGTGAATCTGGGCTTATGGGA |
| RAC1-R | GGAGGTTATATCCTTACCGTACG |
| RHOA F | TGTGTCCCAACGTGCCCATCAT |
| RHOA-R | CTGCCTTCTTCAGGTTTCACCG |
| ROCK-F | GAAACAGTGTTCCATGCTÅGACG |
| ROCK-R | GCCGOTTATTTGATTCCTGCTCC |
| PAK1-F | GTGAAGGCTGTGTCTGAGACTC |
| PAK1-R | GGAAGTGGTTCAATCACAGACCG |
| PAK2-F | CGACTOCAACACAGTGAAGCAG |
| PAK2-R | TCACTACTGCGGGTGCTTCTGT |
| FOXO1-F | CTACGAGTGGATGGTCAAGAGC |
| FOXO1-R | CCAGTTCCTTCATTCTGCACACG |
| Mouse | |
| Slc6a14-F | GCTTGCTGGTTTGTCATCACTCC |
| Slc6a14-R | TACACCAGCCAAGAGCAACTCC |
| myh9-F | CACTACCAACCTCATGGAAGAGG |
| myh9-R | TCCAACTCCTGCGTCTGCTTCT |
| mylk-F | CACTGTCACCTGGTCGCTGAAT |
| mylk-R | GCGCTGTTCTTGGCTACACACT |
| rac1-F | GGACACCATTGAGAAGCTGAAGG |
| rac1-R | GTCTTGAGTCCTCGCTGTGTGA |
| rhoa-F | CTTCAGCAAGGACCAGTTCCCA |
| rhoa-R | GGCGGTCATAATCTCCTGTCC |
| rock-F | CACGCCTAACTGACAAGCACCA |
| rock-R | CAGGTCAACATCTAGCATGGAAC |
| pak1-F | ATTGCTCCACGCCCAGAACACA |
| pak1-R | AAGCATCTGGCGGAGTGGTGTT |
| pak2-F | GCTGTAGTGACAGAGGAAGATGA |
| pak2-R | TCACCAACTGGAGCAGGAATGG |
| foxo1-F | CTACGAGTGGATGGTGAAGAGC |
| foxo1-R | CCAGTTCCTTCATTCTGCACTCG |
| ChIP primers | |
| MYH9-F1 | 5′-CCACCACGTCCAGCT-3′ |
| MYH9-R1 | 5′-CCCTGTTTCTGGAAAAAATAAAAAT-3′ |
| MYH9-F2 | 5′-ATTTTTATTTTTTCCAGAAACAGGG-3′ |
| MYH9-R2 | 5′-GGAAGAGCAAATTTATAGAATGCA-3′ |
| MYH9-F3 | 5′-AGCATTCTATAAATTTGCTCTTCC-3′ |
| MYH9-R3 | 5′-AAGACCATCCTGGGTAACAT-3′ |
| MYH9-F4 | 5′-ATGTTACCCAGGATGGTCTT-3′ |
| MYH9-R4 | 5′-GTTCTTTGCAACATTGTTTATAGTC-3′ |
| MYH9-F5 | 5′-GACTATAAACAATGTTGCAAAGAAC-3′ |
| MYH9-R5 | 5′-CAGGAGATCGAGACCATCC-3′ |
| MYH9-F6 | 5′-GGATGGTCTCGATCTCCTG-3′ |
| MYH9-R6 | 5′-AAAAAGCCATAAGAAATGCAAAC-3′ |
| MYH9-F7 | 5′-GTTTGCATTTCTTATGGCTTTTT-3′ |
| MYH9-R7 | 5′-GCTGAGGCAGGAGAATCG-3′ |
| MYH9-F8 | 5′-CGATTCTCCTGCCTCAGC-3′ |
| MYH9-R8 | 5′-AACTCTGTGACAAATTACTATCAG-3′ |
| MYH9-F9 | 5′-CTGATAGTAATTTGTCACAGAGTT-3′ |
| MYH9-RB | 5′-ACTTGAAGCTGTTTCTGACTT-3′ |
| MYH9-F10 | 5′-AAGTCAGAAACAGCTTCAAGT-3′ |
| MYH9-R10 | 5′-CATGTCATTAACTCGCATACAT-3′ |
| MYH9-F11 | 5′-ATGTATGCGAGTTAATGACATG-3′ |
| MYH9-R11 | 5′-CATCCCACCACAAGGACA-3′ |
| MYH9-F12 | 5′-TGTCCTTGTGGTGGGATG-3′ |
| MYH9-R12 | 5′-TCTAGGGCGGGTCCAA-3′ |
| MYH9-F13 | 5′-TTGGACCCGCCCTAGA-3′ |
| MYL9-R13 | 5′-GGGGCACAATCCCGC-3′ |
| MYLK-F1 | 5′-TCTAGTAGAGGACAAGCTTTCA-3′ |
| MYLK-R1 | 5′-ATATTAATACTACCATCATCAGTTTT |
| | G-3′ |
| MYLK-F2 | 5′-TCAAAACTGATGATGGTAGTATTAATA |
| | T-3′ |
| MYLK-R2 | 5′-CAATAGACAGACGTACTAAAGAATA |
| | C-3′ |
| MYLK-F3 | 5′-GTATTCTTTAGTAGCTCTGTCTATT |
| | G-3′ |
| MYLK-R3 | 5′-GATTATAGGCACGTGCCAC-3′ |
| MYLK-F4 | 5′-GTGGCACGTGCCTATAATC-3′ |
| MYLK-R4 | 5′-ATCTGTACATTTGCTGCCTC-3′ |
| MYLK-F5 | 5′-GAGGCAGCAAATGTACAGAT-3′ |
| MYLK-R5 | 5′-TCTACGATATTAGAGCTCAAATTGT-3′ |
| MYLK-F6 | 5′-GACAATTTGAGCTCTAATATCGTAG-3′ |
| MYLK-R6 | 5′-GCAAAATCATGGTATTCTAATAATT-3′ |
| MYLK-F7 | 5′-TCTCCTTATCCACTTATGAACTAAT-3′ |
| MYLK-R7 | 5′-ATGTTTCTCTGTCCTCTGTATTGAC-3′ |
| MYLK-F8 | 5′-CTGTCAATACAGAGGACAGAGAAAC-3′ |
| MYLK-R8 | 5′-TAACCTGACTTTCATGTAGTCACTT-3′ |
| MYLK-F9 | 5′-ACATGAAAGTCAGGTTAGTGG-3′ |
| MYLK-R9 | 5′-AAAAAAACTGTTTACTGCAGGTT-3′ |
| MYLK-F10 | 5′-AACCTGCAGTAAACAGTTTTTTT-3′ |
| MYLK-R10 | 5′-TTGTGTGTGTTAAGAATCTCTCATT-3′ |
| MYLK-F11 | 5′-ATGAGAGATTCTTAACACACACAAA-3′ |
| MYLK-R11 | 5′-GAAGGTTGTCTATGTAGTTTTTTTC-3′ |
| MYLK-F12 | 5′-TCCAGCACTAAAACTTTAGCC-3′ |
| MYLK-R12 | 5′-AATCCGCAGCCAAACCTA-3′ |
| MYLK-F13 | 5′-TAGGTTTGGCTGCGGATT-3′ |
| MYLK-R13 | 5′-CGCGAGGCGCGTC-3′ |
| Human shANAs | |
| shSLO6A14#1 | CCGGTCGTCTGGCAAGGTGGTATATCTCGAG |
| (TRON0000421162) | ATATACCACCT [symbol] GAGGATTTTTTG |
| shSLO6A14#2 | CCGGGCAATTTCATAGACCTATTACTCGAGT |
| (TRON0000429309) | AATTAGGTAAATTGOTTTTTTG |
| shFOXO1#1 | CCGGATCTACGAGTGGATGGTCAACTCGAGT |
| (TRON0000010333) | TGACCATC [symbol] GTAGATCTTTTTG |
| shFOXO1#2 | CCGGGCCGGAGTTTAGCCAGTCCAACTCGAG |
| (TRON0000039582) | TTGGACTGACTCCGGCTTTTTG |
| ShCTRL | CCGGTGCTTATTGGTATTGTCTCCTCTCGAG |
| (TRON0000003651) | AGGAGACAAAATAAGGATTTTTG |
| Mouse shRNAs | |
| shSIc6a14#1 | CCGGGCTSGAATTTACTGGGTTCATCTCGAG |
| (TRON0000079679) | ATGAACCC [symbol] ATTCCAGCTTTTTG |
| shSIc6a14#2 | CCGGCTGGAATTTACTGGGTTCATTCTCGGA |
| (TRON0000079681) | ATGAACC [symbol] AATTCCAGTTTTTG |
| shFoxo1#1 | CCGGCCCAGTCCAAACTACTCAAAGCTCGAG |
| (TRON0000233396) | CTTTGAGTGGACTGGGTTTTTG |
| shFoxo1#2 | CCGGOGGAGGATTGAACCAGTATAACTOGAG |
| (TRON0000233397) | TTATACTG [symbol] ATCCTOOGTTTTTG |
| shCtrl | CCGGGCATTACCGATGCCATGATATCTCGAG |
| (TRON0000087723) | ATATCATG [symbol] GGTAATGCTTTTTG |

qPCR, quantitative polymerase chain reaction: F, forward; R, reverse
ChIP, chromatin immunoprecipitation; F, forward; R, reverse
shRNA, small hairpin RNA [symbol]
indicates data missing or illegible when filed

TABLE 7
Antibodies

| Antibodies | Source | Identifier |
|---|---|---|
| Immune blot | | |
| anti-SLC6A14 | abcam | ab9 [symbol] 102; RRID: AB_10 [symbol] 96963 |
| anti-SLC6A14 | Sigma | HPA003193; RRID: AB_2190122 |
| anti-HIF-1α | Cell Signaling | #36169; RRID: AB_2799096 |
| | Technology | |
| anti-SLC6A19 | Sigma | PA5-72308; RRID: AB_ 2718162 |
| anti-SLC7A9 | Sigma | PA5-110394 |
| anti-MYH9 | Cell Signaling | #3403; RRID: AB_2147297 |
| | Technology | |
| anti-MYLK | Santa Cruz | sc-365352; RRID: AB_10850176 |
| | Biotechnology | |
| anti-ACTIN | Cell Signaling | #12620; RRID: AB_2797972 |
| | Technology | |
| anti-FOXO1 | Cell Signaling | #2880; RRID: AB_210649 [symbol] |
| | Technology | |
| anti-AKT | Cell Signaling | #4685; RRID: AB_2225340 |
| | Technology | |
| anti-p-AKT | Cell Signaling | #4080; RRID: AB_2315049 |
| | Technology | |
| anti-CA9 | Cell Signaling | #5649; RRID: AB_10706255 |
| | Technology | |
| FACS | | |
| anti-CD3e | Invitrogen | 16-0031-82; RRID: AB_468847 |
| anti-CD8a | Invitrogen | 17-0081-82; RRID: AB_469335 |
| anti-CD90.2 | BD Biosciences | 5 [symbol] 3004; RRID: AB_394543 |
| anti-granzyme B | BD Biosciences | 561142; |
| anti-TNFα | BD Biosciences | 560658, RRID: AB_1727577 |
| anti-IFNγ | BD Biosciences | 563731; RRID: AB_2738391 |
| anti-H-2Kb | BD Biosciences | 5 [symbol] 33570; RRID: AB_394928 |

[symbol]

indicates data missing or illegible when filed

### REFERENCES

1 Semenza, G. L. Hypoxia-inducible factors in physiology and medicine. Cell 148, 399-408 (2012).
2 Altman, B. J., Stine, Z. E. & Dang, C. V. From Krebs to clinic: glutamine metabolism to cancer therapy. Nat Rev Cancer 16, 749 (2016).
3 Sun, R. C. & Denko, N. C. Hypoxic regulation of glutamine metabolism through HIF1 and SIAH2 supports lipid synthesis that is necessary for tumor growth. Cell Metab 19, 285-292 (2014).
4 Wirtz, D., Konstantopoulos, K. & Searson, P. C. The physics of cancer: the role of physical interactions and mechanical forces in metastasis. Nat Rev Cancer 11, 512-522 (2011).
5 Liu, J. et al. Soft fibrin gels promote selection and growth of tumorigenic cells. Nat Mater 11, 734-741 (2012).
6 Ma, J. et al. Reversing drug resistance of soft tumor-repopulating cells by tumor cell-derived chemotherapeutic microparticles. Cell Res 26, 713-727 (2016).
7 Korkaya, H., Liu, S. & Wicha, M. S. Breast cancer stem cells, cytokine networks, and the tumor microenvironment. J Clin Invest 121, 3804-3809 (2011).
8 Cui, T. X. et al. Myeloid-derived suppressor cells enhance sternness of cancer cells by inducing microRNA101 and suppressing the corepressor CtBP2. Immunity 39, 611-621 (2013).
9 Kryczek, I. et al. IL-22(+)CD4(+) T cells promote colorectal cancer stemness via STAT3 transcription factor activation and induction of the methyltransferase DOT1L. Immunity 40, 772-784 (2014).
10 Zou, W., Wolchok, J. D. & Chen, L. PD-L1 (B7-H1) and PD-1 pathway blockade for cancer therapy: Mechanisms, response biomarkers, and combinations. Sci Transl Med 8, 328rv324 (2016).
11 Topalian, S. L., Drake, C. G. & Pardoll, D. M. Immune checkpoint blockade: a common denominator approach to cancer therapy. Cancer Cell 27, 450-461 (2015).
12 Zou, W. Immunosuppressive networks in the tumour environment and their therapeutic relevance. Nat Rev Cancer 5, 263-274 (2005).
13 Sade-Feldman, M. et al. Resistance to checkpoint blockade therapy through inactivation of antigen presentation. Nat Commun 8, 1136 (2017).
14 Zaretsky, J. M. et al. Mutations Associated with Acquired Resistance to PD-1 Blockade in Melanoma. N Engl J Med 375, 819-829 (2016).
15 Shin, D. S. et al. Primary Resistance to PD-1 Blockade Mediated by JAK1/2 Mutations. Cancer Discov 7, 188-201 (2017).
16 Gao, J. et al. Loss of IFN-gamma Pathway Genes in Tumor Cells as a Mechanism of Resistance to Anti-CTLA-4 Therapy. Cell 167, 397-404 e399 (2016).
17 Spranger, S., Bao, R. & Gajewski, T. F. Melanoma-intrinsic beta-catenin signalling prevents anti-tumour immunity. Nature 523, 231-235 (2015).
18 Sheng, W. et al. LSD1 Ablation Stimulates Anti-tumor Immunity and Enables Checkpoint Blockade. Cell 174, 549-563 e519 (2018).
19 Peng, D. et al. Epigenetic silencing of TH1-type chemokines shapes tumour immunity and immunotherapy. Nature 527, 249-253 (2015). 15
20 Manguso, R. T. et al. In vivo CRISPR screening identifies Ptpn2 as a cancer immunotherapy target. Nature 547, 413-418 (2017).
21 Pan, D. et al. A major chromatin regulator determines resistance of tumor cells to T cell-mediated killing. Science 359, 770-775 (2018).
22 Patel, S. J. et al. Identification of essential genes for cancer immunotherapy. Nature 548, 537-542 (2017).
23 Facciabene, A. et al. Tumour hypoxia promotes tolerance and angiogenesis via CCL28 and T(reg) cells. Nature 475, 226-230 (2011).
24 Chiu, D. K. et al. Hypoxia inducible factor HIF-1 promotes myeloid-derived suppressor cells accumulation through ENTPD2/CD39L1 in hepatocellular carcinoma. Nat Commun 8, 517 (2017).
25 Zhang, L. et al. Intratumoral T cells, recurrence, and survival in epithelial ovarian cancer. N Engl J Med 348, 203-213 (2003).
26 Pages, F. et al. International validation of the consensus Immunoscore for the classification of colon cancer: a prognostic and accuracy study. Lancet 391, 2128-2139 (2018).
27 Wang, W. et al. CD8(+) T cells regulate tumour ferroptosis during cancer immunotherapy. Nature 569, 270-274 (2019).
28 Martinez-Lostao, L., Anel, A. & Pardo, J. How Do Cytotoxic Lymphocytes Kill Cancer Cells? Clin Cancer Res 21, 5047-5056 (2015).
29 Basu, R. et al. Cytotoxic T Cells Use Mechanical Force to Potentiate Target Cell Killing. Cell 165, 100-110 (2016).
30 Thiery, J. et al. Perform pores in the endosomal membrane trigger the release of endocytosed granzyme B into the cytosol of target cells. Nat Immunol 12, 770-777 (2011).
31 Morotti, M. et al. Hypoxia-induced switch in SNAT2/SLC38A2 regulation generates endocrine resistance in breast cancer. Proc Natl Acad Sci USA 116, 12452-12461 (2019).
32 Lang, X. et al. Radiotherapy and Immunotherapy Promote Tumoral Lipid Oxidation and Ferroptosis via Synergistic Repression of SLC7A11. Cancer Discov 9, 1673-1685 (2019).
33 Geiger, R. et al. L-Arginine Modulates T Cell Metabolism and Enhances Survival and Anti-tumor Activity. Cell 167, 829-842 e813 (2016).
34 Bian, Y. et al. Cancer SLC43A2 alters T cell methionine metabolism and histone methylation. Nature, in press (2020).
35 Kandasamy, P., Gyimesi, G., Kanai, Y. & Hediger, M. A. Amino acid transporters revisited: New views in health and disease. Trends Biochem Sci 43, 752-789 (2018).
36 Hagglund, M. G. et al. Characterization of the transporterB0AT3 (Slc6a17) in the rodent central nervous system. BMC Neurosci 14, 54 (2013).
37 Sun, C. et al. Spatially resolved metabolomics to discover tumor-associated metabolic alterations. Proc Natl Acad Sci USA 116, 52-57 (2019).
38 Liu, Y. et al. Visualization of perforin/gasdermin/complement-formed pores in real cell membranes using atomic force microscopy. Cell Mol Immunol 16, 611-620 (2019).
39 Yousafzai, M. S. et al. Investigating the effect of cell substrate on cancer cell stiffness by optical tweezers. J Biomech 60, 266-269 (2017).
40 Dogterom, M. & Koenderink, G. H. Actin-microtubule crosstalk in cell biology. Nat Rev Mol Cell Biol 20, 38-54 (2019). 16
41 Karunakaran, S. et al. Interaction of tryptophan derivatives with SLC6A14 (ATB0,+) reveals the potential of the transporter as a drug target for cancer chemotherapy. Biochem J 414, 343-355 (2008).
42 Rafiq, N. B. M. et al. A mechano-signalling network linking microtubules, myosin IIA filaments and integrin-based adhesions. Nat Mater 18, 638-649 (2019).
43 Kilberg, M. S., Balasubramanian, M., Fu, L. & Shan, J. The transcription factor network associated with the amino acid response in mammalian cells. Adv Nutr 3, 295-306 (2012).
44 Tang, Z. et al. GEPIA: a web server for cancer and normal gene expression profiling and interactive analyses. Nucleic Acids Res 45, W98-W102 (2017).
45 Fornes, O. et al. JASPAR 2020: update of the open-access database of transcription factor binding profiles. Nucleic Acids Res 48, D87-D92 (2020).
46 Surcel, A. et al. Pharmacological activation of myosin II paralogs to correct cell mechanics defects. Proc Natl Acad Sci USA 112, 1428-1433 (2015).
47 van Meijl, L. E., Popeijus, H. E. & Mensink, R. P. Amino acids stimulate Akt phosphorylation, and reduce IL-8 production and NF-kappaB activity in HepG2 liver cells. Mol Nutr Food Res 54, 1568-1573 (2010).
48 Lam, E. W., Brosens, J. J., Gomes, A. R. & Koo, C. Y. Forkhead box proteins: tuning forks for transcriptional harmony. Nat Rev Cancer 13, 482-495 (2013).
49 Harel, M. et al. Proteomics of Melanoma Response to Immunotherapy Reveals Mitochondrial Dependence. Cell 179, 236-250 e218 (2019).
50 Schulze, A. & Harris, A. L. How cancer metabolism is tuned for proliferation and vulnerable to disruption. Nature 491, 364-373 (2012).
51 Knott, S. R. V. et al. Asparagine bioavailability governs metastasis in a model of breast cancer. Nature 554, 378-381 (2018).
52 Gao, X. et al. Dietary methionine influences therapy in mouse cancer models and alters human metabolism. Nature 572, 397-401 (2019).
53 Tato, I., Bartrons, R., Ventura, F. & Rosa, J. L. Amino acids activate mammalian target of rapamycin complex 2 (mTORC2) via PI3K/Akt signaling. J Biol Chem 286, 6128-6142 (2011).
54 Moloughney, J. G. et al. mTORC2 Responds to Glutamine Catabolite Levels to Modulate the Hexosamine Biosynthesis Enzyme GFAT1. Mol Cell 63, 811-826 (2016).
55 Sun, K., Wu, Z., Ji, Y. & Wu, G. Glycine Regulates Protein Turnover by Activating Protein Kinase B/Mammalian Target of Rapamycin and by Inhibiting MuRF1 and Atrogin-1 Gene Expression in C2C12 Myoblasts. J Nutr 146, 2461-2467 (2016).
56 Vicente-Manzanares, M., Ma, X., Adelstein, R. S. & Horwitz, A. R. Non-muscle myosin II takes centre stage in cell adhesion and migration. Nat Rev Mol Cell Biol 10, 778-790 (2009).
57 Dupont, S. et al. Role of YAP/TAZ in mechanotransduction. Nature 474, 179-183 (2011).
58 Meng, Z. et al. RAP2 mediates mechanoresponses of the Hippo pathway. Nature 560, 655-660 (2018).
59 Plodinec, M. et al. The nanomechanical signature of breast cancer. Nat Nanotechnol 7, 757-765 (2012). 17
60 Karunakaran, S. et al. SLC6A14 (ATB0,+) protein, a highly concentrative and broad specific amino acid transporter, is a novel and effective drug target for treatment of estrogen receptor-positive breast cancer. J Biol Chem 286, 31830-31838 (2011).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of treating cancer, comprising:
administering to a subject an agent that inhibits one or more activities of Solute Carrier Family 6 Member 14 (SLC6A14) in combination with immunotherapy.

2. A method of treating cancer, comprising:
a) assaying a sample from a subject diagnosed with cancer for the level of expression of SLC6A14;
b) identifying said subject as having increased levels of expression of SLC6A14 relative to a control level; and
b) administering an agent that inhibits one or more activities of SLC6A14 to said subject.

3. The method of claim 1, wherein said agent is an antibody that binds to SLC6A14.

4. The method of claim 1, wherein said antibody is a monoclonal antibody.

5. The method of any of the preceding claims, wherein said monoclonal antibody is humanized.

6. The method of claim 1, wherein said agent is selected from the group consisting of a nucleic acid and a small molecule.

7. The method of claim 1, wherein said nucleic acid is selected from the group consisting of a shRNA, a miRNA, and an antisense RNA.

8. The method of claim 7, wherein said small molecule is α-MT.

9. The method of claim 1, wherein said cancer overexpresses SLC6A14.

10. The method of claim 1, wherein said cancer is selected from the group consisting of breast, lung, bladder, cervical, colon, head and neck, Hodgkin lymphoma, liver, renal cell, skin, stomach, and rectal.

11. The method of claim 10, wherein said cancer is breast cancer.

12. The method of claim 1, wherein said method further comprises administering a second cancer therapy to said subject.

13. The method of claim 12, wherein said second cancer therapy is chemotherapy and/or immunotherapy.

14. The method of claim 13, wherein said immunotherapy is selected from the group consisting of CAR-T therapy, TCR therapy, antibody immunotherapy, and checkpoint inhibitors.

15. The method of claim 14, wherein said checkpoint inhibitor is selected from the group consisting of ipilimumab, nivolumab, pembrolizumab, and atezolizumab.

16-17. (canceled)
